(19) United States
(12) Patent Application Publication
(10) Pub. No.: US 2016/0193151 A1
(43) Pub. Date: Jul. 7, 2016

(54) DOSAGE FORM INCORPORATING AN AMORPHOUS DRUG SOLID SOLUTION

(71) Applicants: Maria del Pilar Noriega Escobar, Medellin (CO); Laura Restrepo Uribe, Medellin (CO); Marco Enrique Sanjuan Mejia, Barranquilla (CO); Carlos Arturo Salazar Altamar, Barranquilla (CO); Claudia Andrea Silva Blanco, Barranquilla (CO)

(72) Inventors: Maria del Pilar Noriega Escobar, Medellin (CO); Laura Restrepo Uribe, Medellin (CO); Marco Enrique Sanjuan Mejia, Barranquilla (CO); Carlos Arturo Salazar Altamar, Barranquilla (CO); Claudia Andrea Silva Blanco, Barranquilla (CO)

(21) Appl. No.: 14/849,571

(22) Filed: Sep. 9, 2015

Related U.S. Application Data
(60) Provisional application No. 62/100,117, filed on Jan. 6, 2015.

(51) Int. Cl. A61K 9/16; A61K 31/554; A61K 31/216; A61K 9/70; A61K 9/20; A61K 9/06; A61K 9/00; A61K 31/192; A61K 9/48

(52) U.S. Cl. CPC A61K 9/1694; A61K 31/192; A61K 31/554; A61K 31/216; A61K 9/4825; A61K 9/2095; A61K 9/06; A61K 9/0014; A61K 9/7023

(57) ABSTRACT

The dissolution of Active Pharmaceutical Ingredients in polymeric melts plays an important role in the manufacturing of drugs that use polymers as excipients. The dissolution kinetics is essential for designing the processing equipment, describing the operating conditions, and defining material properties, for example, the appropriate API-polymer(s) pair. In one embodiment of the invention, the solubility of ketoprofen (KTO) in Soluplus®, Kollidon® VA64, Kollidon® SR and a combination of three; was analyzed under Hot Melt Extrusion (HME) processing conditions. Thermal characterization techniques show that a single phase amorphous solid solution (only one Tg) was achieved by HME at 120° C. and 70 rpm. The sample's stability was analyzed for 4 weeks and the single phase amorphous solid solution was maintained during that time. An extended release profile of KTO was achieved, releasing 100% of KTO in 12 h. The invention is particularly useful to target a specific release profile.

This application claims the priority benefit under 35 U.S.C. section 119 of U.S. Provisional Patent Application No. 62/100,117 entitled “Dosage Form Incorporating An Amorphous Drug Solid Solution” filed on Jan. 6, 2015; which is in its entirety herein incorporated by reference.

### FIELD OF THE INVENTION

The present invention relates to pharmaceutical compositions comprising one or more therapeutic agents in amorphous solid solution form.

This invention generally relates to orally bioavailable solid dosage forms of poorly water-soluble pharmaceutical agents. The present invention provides processes for making and forms of solid solutionns of pharmaceutical active ingredients. The instant invention also relates to a solid amorphous drug composition, and, more particularly, a solid amorphous composition of poorly soluble compounds comprising said compound and suitable polymers.

The present invention features a process for producing active substance compositions in which the active substance is present as a single phase amorphous solid solution in a polymer matrix, and active substance compositions produced thereby.

The invention further relates to a process for producing solid dosage forms by mixing in a melted state at least one polymer and at least one active ingredient to form an amorphous solid solution. The invention particularly relates to a process for producing amorphous solid solutions of pharmaceutical forms.

More particularly, the invention relates to a process for producing an amorphous drug solid solution utilizing a twin-screw extruder, which finds application chiefly in the field of pharmaceutical manufacture.

### BACKGROUND OF THE INVENTION

It has been estimated that more than 60% of Active Pharmaceutical Ingredients (API) in development have poor bioavailability due to low aqueous solubility (Manufacturing chemist, Mar. 2010, 24-25). This percentage is likely to increase in the future with the increased use of combinatorial chemistry in drug discovery targeting lipophilic receptors. Poor bioavailability results in increased development times, decreased efficacy, increased inter- and intra-patient variability and side effects, and high dose that reduce patient compliance and increase cost. Thus, the ability to improve drug solubility and/or dissolution rate and, hence, bioavailability through formulation technology is critical to improve a drug product's efficacy and safety, and reduce its cost. In recent years, one of the major focuses for the pharmaceutical formulators is to identify strategies that would improve the bioavailability of active pharmaceutical ingredients (APIs) by enhancing their dissolution rate and/or solubility. In particular, poorly soluble API's can be changed to amorphous or microcrystalline forms through formulation approaches, which provide a fast dissolution rate and/or higher apparent solubility in the gastric and intestinal fluids.

It is also known that many pharmaceutical agents are such highly complex chemical structures that they are insoluble or only sparingly soluble in water. This results in no or very low dissolution from conventional dosage forms designed for oral administration. Low dissolution rates results in no or very little bioavailability of the active chemical substance, thus making oral delivery ineffective therapeutically, and necessitating parenteral administration in order to achieve a beneficial therapeutic result. Drug products that are limited to parenteral delivery leads to increased costs of medical care, due to higher costs of manufacturing, more costly accessories required for delivery, and in many cases hospitalization of the patient to ensure proper dosing (e.g., sterile intravenous delivery).

Poorly water-soluble drugs that undergo dissolution rate-limited gastrointestinal absorption generally show increased bioavailability when the rate of dissolution is improved. To enhance the dissolution property and potentially the bioavailability of poorly water-soluble drugs, many strategies and methods have been proposed and used, which include particle size reduction, salt selection, formation of molecular complexes and solid dispersions, and the use of metastable polymorphic forms, co-solvents, and surface-active agents. Of these methods, the use of surface-active agents is mainly to improve the wettability of poorly water-soluble drugs, which eventually results in the enhancement of the rate of dissolution.

The pharmaceutical industry is facing two main problems, poorly soluble drugs that require an increased dosage formulation so the drug absorption can be guaranteed, and the low bioavailability of the drug due to inefficient dissolution during its passage through the gastrointestinal tract (Niu et al., 2013). Different approaches can be applied to overcome the solubility and bioavailability problems, one of them the manufacturing of solid dispersions (Kolter et al., 2012; Prodduturi et al., 2007).

Hot Melt Extrusion (HME) is a recognized process that has been used in the last two decades in the pharmaceutical field and has become very popular because it is a continuous process, solvent free, easy to clean and can be used for the preparation of different drug delivery systems; including granules, pellets, sustained released tablets, suppositories, stents, ophthalmic inserts, and transdermal and transmucosal delivery systems (Djuris et al., 2013; Prodduturi et al., 2007). Since it is a continuous process, fewer steps are involved resulting in reduced production cost. There are different types of solid dispersions (Dhirendra et al., 2009; Shah et al., 2013), but only 3 can be achieved by HME crystalline solid dispersion, amorphous solid dispersion, and solid solutions (Sarode et al., 2013; Shah et al., 2013). Crystalline solid dispersion is a system in which the crystalline Active Pharmaceutical Ingredient (API) is dispersed into an amorphous polymer matrix. The Differential Scanning calorimetry (DSC) profile for such a system is characterized by the presence of a melting point (Tm) corresponding to the crystalline API and a characteristic glass transition temperature (Tg) corresponding to the amorphous polymer excipient. Amorphous solid dispersions result when a melt extruded API-polymer excipient is cooled at a rate that does not allow the drug to recrystallize or processed at temperatures where the API melts but remains immiscible with the polymer excipient. The DSC profile for amorphous solid dispersions is characterized by the presence of two Tg. They can be unstable because the API can revert to the more stable crystalline form. In the solid solution, the API molecules are molecularly dispersed in the polymeric matrix and exhibit a single Tg. This system is more stable and has a longer shelf life (jan et al., 2012; Shah et al., 2013).

It has been shown that the dissolution behavior of HME solid dispersion depends on the physicochemical characteristics of the excipient(s) selected, therefore, the choice of excipients plays an important role in a successful formulation (Kalivoda et al., 2012; Yang et al., 2011). Different polymer excipients can be employed to prepare immediate and sustained release profiles via HME. Vinylpyrrolidone-vinylacetate copolymer (Kollidon® VA64) and polyvinylcaprolactam-polyvinyl acetate-polyethylene glycol graft co-polymer (Soluplus®) have been applied as polymer excipients for immediate release (IR) profiles. On the other hand, polyvinyl acetate-polyvynilpyrrolidone (Kollidon® SR) has been applied as polymer excipient for sustained release (SR) profiles (Almeida et al., 2012; Kolivoda et al., 2012; Kollidon S R—Technical Information 2011; Yang et al., 2010). Moreover, Soluplus® has been shown to increase the drug absorption through the intestinal wall when applied as a solid solution (Jan et al., 2012).

Ketoprofen (KTO) is a nonselective NonSteroidal Anti-Inflammatory Drug (NSAID). Its chemical name is 2-(3-benzoylphenyl)-propionic acid. It has analgesic, anti-inflammatory and antipyretic effects and is more used in the treatment of acute and long-term rheumatoid arthritis, osteoarthritis and ankylosis spondylitis (Dixit et al., 2013; Jan et al., 2012; Shoin et al., 2012; Vueba et al., 2006; Vueba et al., 2004). Ketoprofen is classified by the Biopharmaceutical Classification System (BCS) as a class II drug because of its high permeability and poorly water-soluble properties (Fukudaa et al., 2008; Shoin et al., 2012; Yadav et al., 2013). Based on these characteristics, KTO is a good candidate for improving its dissolution profile, solubility and bioavailability by HME.

The present invention provides KTO and other drugs dissolution kinetics in different polymeric melts (IR and SR excipients) combining static and dynamic characterization methods for HME applications and drug release profiles. The most suitable blend of API and excipient(s) (IR/SR polymer excipient(s)) can improve the drug release profile (Maschke et al., 2011), and in combination with HME. A more sustained release can be achieved because the blends are less porous and have better mechanical properties (Yang et al., 2010). A better understanding of the solid dispersion, particularly the existing physical form of a drug in the polymer excipient is necessary to predict the stability, solubility and hence bioavailability of melt extrudates.

### SUMMARY OF THE INVENTION

The present invention provides a method for making a dosage form comprising: (a) preparing by melt extrusion an amorphous solid solution of one or more active ingredient; and (b) placing said amorphous solid solution of said active ingredient in a suitable dosage form.

The invention also provides a process for preparing a solid amorphous solution compositions comprising the steps of: (a) preparing a homogenous blend of (i) at least one active pharmaceutical ingredient (API) which belongs to BCS class II and/or IV; and (ii) one or more water-soluble physiologically acceptable polymers; (b) heating, mixing and/or kneading the resultant blend of step (a) through an extruder to result in a homogenous melt and/or granulation; (c) forcing the resultant melt obtained in step (b) through one or more orifices, nozzles, or moulds; (d) cooling the extrudate of step (c) by means of air to yield an amorphous solid solution; and (e) optionally, grinding or milling the solid solution obtained in step (d).

The invention is further directed to stable binary and ternary amorphous solid solution compositions with enhanced bioavailability comprising: (a) about 1% wt. to about 50% wt. of one or more poorly soluble active pharmaceutical ingredient (API) which belong to Biopharmaceutics Classification System (BC S) class II and/or IV; (b) about 50% wt. to about 99% wt. of at least one Physiologically acceptable polymer and wherein the amorphous solid solution is capable of inhibiting crystallization of API in said amorphous solid state and/or Bioequivalent aqueous medium.

The instant invention also relates to a method for enhancing the bioavailability of an active ingredient in a mammal, which method comprises administering an effective amount of a amorphous solid solution of an active ingredient to said mammal.

The invention is also directed to a milled amorphous solid solution of an active ingredient.

The invention further provides a dosage form incorporating an amorphous solid solution of an active ingredient.

The invention also relates to a process for producing an amorphous single phase solid solution of a drug dissolved in a polymer carrier or diluent which comprises passing a mixture comprising said drug and said polymer through a twin screw compounding extruder having retaining barrels, with said twin screw compounding extruder being equipped with paddle means on each of two screw shafts, whereby said mixture passes between said paddle means and is sheared and compounded thereby, and operating said twin screw extruder while sufficiently heating the barrels to obtain an extrudate in the form of said solid solution and wherein said heating is to a temperature below the decomposition temperature of the drug or polymer.

### BRIEF DESCRIPTION OF THE FIGURES

FIG. 1 is a general flow diagram illustrating the method of making solid solutions by hot melt extrusion and by kneading.

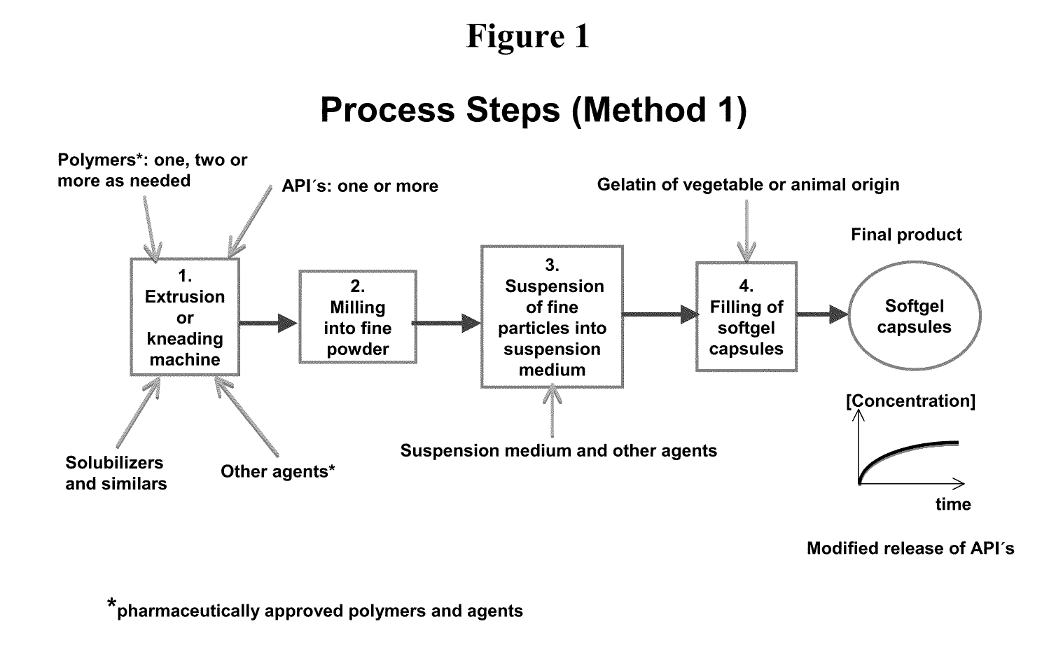

FIG. 2 shows a soft gel capsule having the Hot Melt Extrusion solid solution in particle form inside the capsule.

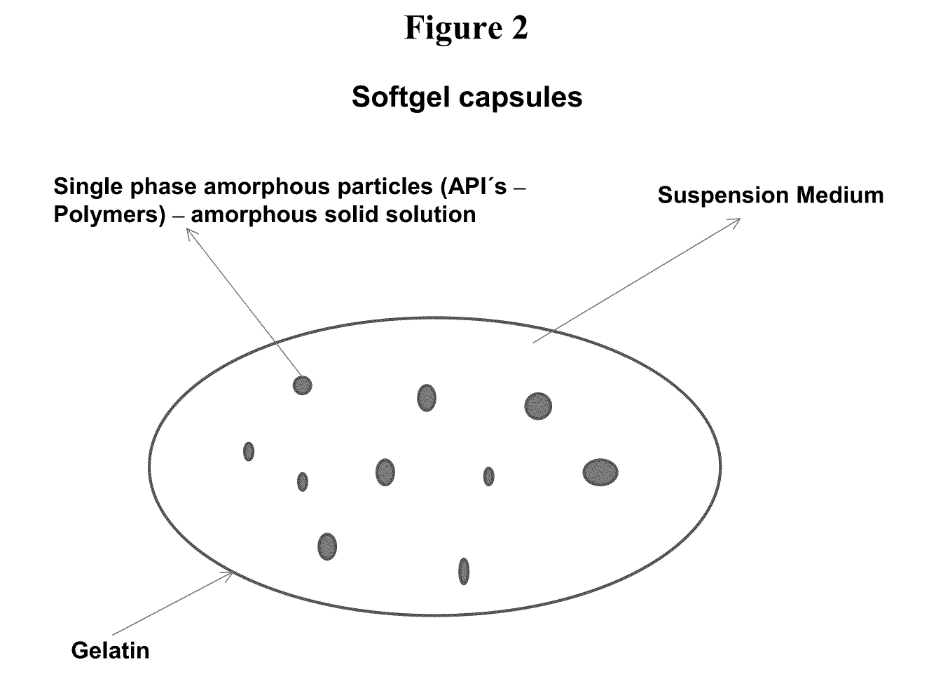

FIG. 3 is an alternate method for making the solid solutions of the invention and further processing thereof.

FIG. 4 features another method for making the products of the invention using Nutraceuticals.

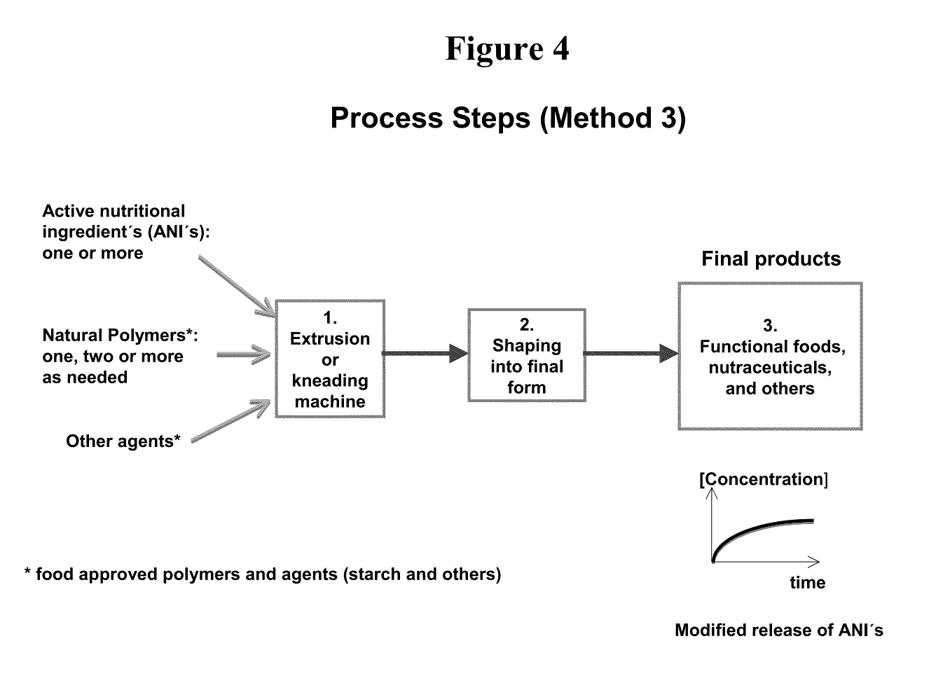

FIG. 5A shows differential scanning calorimetry (DSC) during second heating of binary samples consisting of KTO and Soluplus® and KTO and Kollidon® SR from film casting.

FIG. 5B shows DSC during second heating of binary samples consisting of KTO and Kollidon® VA64 from film casting.

FIG. 6A describes DSC second heating of ternary samples containing 20, 35 and 50% KTO in Soluplus®-Kollidon® SR from film casting.

FIG. 6B illustrates DSC second heating of ternary samples containing 20, 35 and 50% KTO in Kollidon® VA64-Kollidon® SR.

FIG. 7 shows DSC second heating from samples prepared by Haake rheometer.

FIG. 8 describes the solubility characteristics of KTO in Soluplus®-Kollidon® SR by rheometry.

FIG. 9A illustrates the release profiles of hot melt extruded samples of Ketoprofen. The rhombi represent the sample with 35% KTO, 33% Soluplus® and 32% Kollidon® SR. The triangles represent the sample with 50% KTO and 50% Soluplus®. The square represent the sample with 50% KTO, 25% Soluplus® and 25% Kollidon® SR.

FIG. 9B features a comparison of release profiles from the literature and the sample containing 35% KTO, 33% Soluplus® and 32% Kollidon® SR (rhombi). The squares represent the release profile of a formulation produced by hot melt extrusion HME with 20% KTO, 35% ETHOCEL Standard 10 Premium and 45% Polyox™ N10 (Coppens et al., 2009). The triangles represent the release profile of a formulation produced by HME with 30% KTO, 50% Eudragit™ E and 20% polyvinylpyrrolidone (PVP) (Gue et al., 2013). The Xs on the far left side represent the release profile of a formulation produce by HME with 50% KTO and 50% sulfobutyl ether β-cyclodextrin (Fukudaa et al., 2008). The asterisks represent the release profile of a formulation produce by HME with 50% KTO and 50% β-cyclodextrin Fukudaa et al., 2008). The circles represent the release profile of a formulation produce by HME with 30% KTO and 70% hydroxypropylcellulose (HPC) (Loreti et al., 2014). It can be seen that the release profile from the sample reported in this paper, is more extended that the ones reported in the literature. The horizontal bars represent the standard deviation.

FIG. 9C illustrates the comparison of KTO release profiles prepared by direct compression (DC) and sample containing 35% KTO, 33% Soluplus® and 32% Kollidon® SR prepared by HME (rhombi). The circles represent the release profile of a formulation produced DC of KTO with PGA-co-Pentadecalantone (10:2). The squares represent the release profile of a formulation produced DC of KTO with Poly(glycolide)PGA (10:2). The triangles represent the release profile of a formulation produced DC of KTO with PGA-co-Copralactonem (10:2). The Xs represent the release profile of a formulation produced DC of KTO with Ethocel FP100 Premium (10:2). The samples prepared by DC in this figure were reported by Jan et al., in 2012. It can be seen that the release profile of samples prepared by HME achieved the 100% release in 12 h, in contrast with samples prepared by DC where the release was not even 80% in 12 h. The horizontal bars represent the standard deviation.

FIG. 10 describes the release profile of blended dosage forms containing Ketoprofen with suitable polymers of the invention.

FIG. 11 shows the release profile of several ketoprofen dosage forms prepared by twin-screw extruder using different processing conditions and suitable polymers of the invention.

FIG. 12 features the release profile of a blended dosage form containing 35% Quetiapine Fumarate, 32% polyvinyl caprolactam-polyvinyl acetate-polyethylene glycol, and 32% hypromellose acetate succinate.

FIG. 13 illustrates the release profile of blended dosage forms containing 50% Fenofibrate, 25% polyvinyl caprolactam-polyvinyl acetate-polyethylene glycol, 25% polyvinyl acetate-polyvinylpyrrolidone prepared under different conditions.

FIG. 14 shows the stability characteristics by Differential Scanning calorimetry of samples prepared by the Haake laboratory mixer.

### DETAILED DESCRIPTION OF THE INVENTION

The instant invention provides a method to enhance the dissolution, bioavailability, release of active ingredients and other properties of poorly water-soluble drugs. The API's (Active pharmaceutical ingredients) belong to the following classification: poorly soluble in water [Biopharmaceutical Classification System: Class II (low solubility and high permeability) and Class IV (low solubility and low permeability)].

The method of the invention can also be used for manufacturing dietary supplements or nutraceuticals or functional foods.

The method of the invention relates to processing of amorphous solid solutions comprising at least one active ingredient and at least one pharmaceutical approved polymer for manufacturing a formulation.

The unique amorphous solid solutions of the invention provide significantly enhanced bioavailability because of superior dissolution of the API resulting from availability of high surface area, high interfacial activity and particle morphology.

The method of manufacturing the amorphous single phase particles is as follows:
(1) Determining most suitable mixing configuration (screws or rotors or others);
(2) Feeding components individually or Pre-blending and metering the formulation,
(3) Heating up and reach optimal processing window to avoid polymorphism:
The temperature of the process should be: Tg amorphous polymer or Tm semicrystalline polymer<Processing Temperature<Tm API. Note flow with increased chain mobility (Polymer).
(4) Shaping and cooling the single phase material system to a desired form.

The hot-melt extrusion process is generally described as follows. An effective amount of a powdered therapeutic compound is mixed with one or more of the pharmaceutically acceptable polymers. In some other embodiments, the therapeutic compound: polymer ratio is generally about 0.01: about 99.99 to about 20: about 80% wt., depending on the desired release profile, the pharmacological activity and toxicity of the therapeutic compound and other such considerations. The mixture is then placed in the extruder hopper and passed through the heated area of the extruder at a temperature which will melt or soften the polymer, to form a matrix throughout which the therapeutic compound is in solution in amorphous form. The molten or softened mixture then exits via a die, or other such element, at which time, the mixture (now called the extrudate) begins to harden. Since the extrudate is still warm or hot upon exiting the die, it may be easily shaped, molded, chopped, ground, milled, molded, spheronized into beads, cut into strands, tableted or otherwise processed to the desired physical form i.e., placed in a capsule. Typical melt extrusion systems capable of carrying-out the present invention include a suitable extruder drive motor having variable speed and constant torque control, start-stop controls, and ammeter. In addition, the system will include a temperature control console which includes temperature sensors, cooling means and temperature indicators throughout the length of the extruder. In addition, the system will include an extruder such as twin-screw extruder which consists of two counter-rotating intermeshing screws enclosed within a cylinder or barrel having an aperture or die at the exit thereof. The feed materials enter through a feed hopper and is moved through the barrel by the screws and is forced through the die into strands which are thereafter conveyed such as by a continuous movable belt to allow for cooling and being directed to a pelletizer or other suitable device to render the extruded ropes into the multiparticulate system. The pelletizer can consist of rollers, fixed knife, rotating cutter and the like.

HME is carried out using an extruder—a barrel containing one or two rotating screws that transport material down the barrel. The extruder typically consist of four distinct parts: An opening though which material enters the barrel, that may have a hopper that is filled with the material(s) to be extruded, or that may be continuously supplied to in a controlled manner by one or more external feeder(s), a conveying section (process section), which comprises the barrel and the screw(s) that transport, and where applicable, mix the material, an orifice (die) for shaping the material as it leaves the extruder and downstream auxiliary equipment for cooling, cutting and/or collecting the finished product.

There are two types of extruders: single and twin screw extruders. Single screw extruders are primarily used for melting and conveying polymers to extrude them into continuous shapes, whereas twin screw extruders are used for melt-mixing polymers with additional materials (i.e., API's). In the production of pharmaceutical formulations, which require homogeneous and consistent mixing of multiple formulation ingredients, a twin screw extruder is preferred because the rotation of the intermeshing screws provides better mixing to produce a homogeneous solid-solution of API in polymer. As shown in the present invention, one can improve the dissolution rate and bioavailability of poorly-water soluble API formulations.

Melting is accomplished by frictional heating within the barrel, and for twin-screw extruders, as the materials undergo shearing between the rotating screws and between the screws and the wall of the barrel as they are conveyed. The barrel is also heated with heaters mounted on the barrel, or cooled with water. The barrel section temperatures are usually optimized so that the viscosity of the melt is low enough to allow conveying down the barrel and proper mixing, while keeping temperatures low enough to avoid thermal degradation of the materials.

The screws of a twin screw-extruder are usually to provide different types of mixing and conveying conditions at various zones in the barrel. The length of the screw in relation to the barrel diameter (the L/D ratio) is chosen to optimize the degree of mixing and the number of zones required to achieve the final product characteristics.

Rotation of the screws creates distributive and homogeneous mixing. This uniformly blends the materials. The use of different mixing elements allows the twin screw extruder to perform both particle-size reduction and mixing so that the APIs can be homogeneously incorporated into the polymer to form a single phase amorphous solid solution.

As with any dosage form, material selection is critical in the development of a successful product. For most applications, the polymer should be thermoplastic, stable at the temperatures used in the process, and chemically compatible with the API during extrusion. For solid oral dosage forms many polymers are available as further disclosed in the present application.

HME allows the API to be mixed with the polymer under the minimum of shear and thermal stresses and hence with the formation of minimal process-related API degradants. Antioxidants may be included within the formulation, and the short residence time in the barrel (typically on the order of minutes) also helps to minimize thermal degradation especially compared to batch mixing and other compounding processes.

The process conditions of the invention using HME are chosen to maximize API mixing with the polymer, while minimizing API degradation.

The method of the invention is conducted below the decomposition temperature of all components of said mixture wherein said mixture is heated, without thermal and/or oxidative degradation.

The hot-melt extrusion process employed in some embodiments of the invention is conducted at an elevated temperature, i.e. the heating zone(s) of the extruder is above room temperature (about 20° C.). It is important to select an operating temperature range that will minimize the degradation or decomposition of the therapeutic compound during processing. The operating temperature range is generally in the range of from about 60° C. to about 190° C. as determined by the setting for the extruder heating zone(s). More specifically, the hot-melt temperature is preferably from 50° to 250° C., more preferably from 60 to 200° C., still more preferably from 90° to 190° C. When the hot-melt temperature is less than 50° C., incomplete melting may impede extrusion. When the hot-melt temperature is more than 250° C., there are possibilities of reduction in molecular weight due to decomposition of the polymer or the drug, and deactivation.

The extrusion conditions are not particularly limited insofar as they permit extrusion of a composition for hot-melt extrusion having preferably a viscosity, during hot-melt extrusion, of from 1 to 100000 Pas. When a uniaxial piston extruder is used, the extrusion rate is preferably from 1 to 1000 mm/min, more preferably from 10 to 500 mm/min. When a twin-screw extruder is used, the screw rotation number is preferably from 1 to 1000 rpm, more preferably from 10 to 500 rpm. When the extrusion rate is less than 1 mm/min or the screw rotation number is less than 1 rpm, the residence time in the extruder becomes long, which may cause thermal decomposition. When the extrusion rate is more than 1000 mm/min or the screw rotation number is more than 1000 rpm, the hot-melt procedure during kneading may become insufficient, which may result in non-uniform molten state of the drug and the polymer in the hot-melt extrusion product.

After the extrusion, the hot-melt extrusion product is cooled after the die outlet port by natural cooling at room temperature (from 1 to 30° C.) or by blowing of cold air. It is desired to rapidly cool the hot-melt extrusion product preferably to a temperature of not higher than 50° C., more preferably to a temperature of not higher than room temperature (not higher than 30° C.) to minimize the thermal decomposition of the drug and to prevent recrystallization when the drug is in an amorphous form.

The hot-melt extrusion product after cooling may be optionally pelletized into pellets of from 0.1 to 5 mm by using a cutter, or optionally ground or milled to regulate the particle size until it becomes granular or powdery. As for grinding, an impact grinder such as a jet mill, a knife mill and a pin mill is preferred because its structure prevents an increase in the temperature of the product therein. When the temperature inside the cutter or grinder becomes high, the HPMCAS is thermally softened and the particles adhere to each other so that it is preferred to grind the extrusion product while blowing cold air.

In the present invention, the resulting extruded product is typically milled into fine powder of a particle size <180 μm.

The process is a dry process, there is no heat dissipation. For some API's, the milling process could be cryogenic. The single amorphous phase particles produced by extrusion or kneading must be maintained.

The resulting fine particles are then placed into suspension medium. The suspension medium viscosity, approx. 1000 cP<μ<2500 cP (55% wt. solids, Particles <180 μm) Suspension medium temperature is approximately. Troom<T<42° C. The stability of the single amorphous phase particles produced by extrusion or kneading and milling must be maintained. The suspension medium may be a lipophilic carrier or lipophilic systems preventing the settling down of fine particles and Pumpable.

The resulting product of the above steps is used for filling of softgel capsules. The soft gelatin capsules are prepared by the rotary die encapsulation method, dropping or others. The gelatin may be from vegetal or animal origin.

According to one embodiment of the present invention, the poorly soluble drugs are BCS Class II drugs having high permeability and low solubility; or Class IV drugs having low permeability and low solubility. The BCS Class II or Class IV API may belong to analgesics, anti-inflammatory agents, anti-helminthics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, erectile dysfunction improvement agents, immune-suppressants, anti-protozoal agents, anti-thyroid agents, anxiolytic agents, sedatives, hypnotics, neuroleptics, (3-blockers, cardiac inotropic agents, corticosteroids, diuretics, anti-parkinsonian agents, gastro-intestinal agents, histamine receptor antagonists, keratolyptics, lipid regulating agents, anti-anginal agents, Cox-2 inhibitors, leukotriene inhibitors, macrolides, muscle relaxants, nutritional agents, opiod analgesics, protease inhibitors, sex hormones, stimulants, muscle relaxants, anti-osteoporosis agents, anti-obesity agents, cognition enhancers, anti-urinary incontinence agents, nutritional oils, anti-benign prostate hypertrophy agents, essential fatty acids, non-essential fatty acids, antipyretics, muscular relaxants, anti-convulsants, anti-emetics, anti-psychotics, and/or anti-Alzheimer agents.

The APIs that belong to BCS Class II are poorly soluble, but are absorbed from the solution by the lining of the stomach and/or intestine. The non-limiting BCS Class II drugs are selected from the group consisting of Albendazole, Acyclovir, Azithromycin, Cefdinir, Cefuroxime axetil, Chloroquine, Clarithromycin, Clofazimine, Diloxanide, Efavirenz, Fluconazole, Griseofulvin, Indinavir, Itraconazole, Ketoconalzole, Lopinavir, Mebendazole, Nelfinavir, Nevirapine, Niclosamide, Praziquantel, Pyrantel, Pyrimethamine, Quinine, Ritonavir, Bicalutamide, Cyproterone, Gefitinib, Imatinib, Tamoxifen, Cyclosporine, Mycophenolate mofetil, Tacrolimus. Acetazolamide, Atorvastatin, Benidipine, Candesartan cilexetil, Carvedilol, Cilostazol, Clopidogrel, Ethylicosapentate, Ezetimibe, Fenofibrate, Irbesartan, Manidipine, Nifedipine, Nisoldipine, Simvastatin, Spironolactone, Telmisartan, Ticlopidine, Valsartan, Verapamil, Warfarin, Acetaminophen, Amisulpride, Aripiprazole, Carbamazepine, Celecoxib, Chlorpromazine, Clozapine, Diazepam, Diclofenac, Flurbiprofen, Haloperidol, Ibuprofen, Ketoprofen, Lamotrigine, Levodopa, Lorazepam, Meloxicam, Metaxalone, Methylphenidate, Metoclopramide, Nicergoline, Naproxen, Olanzapine, Oxcarbazepine, Phenyloin, Quetiapine, Risperidone, Rofecoxib, Valproic acid, Isotretinoin, Dexamethasone, Danazol, Epalrestat, Gliclazide, Glimepiride, Glipizide, Glyburide (glibenclamide), levothyroxine sodium, Medroxyprogesterone, Pioglitazone, Raloxifene, Mosapride, Orlistat, Cisapride, Rebamipide, Sulfasalazine, Teprenone, Ursodeoxycholic Acid, Ebastine, Hydroxyzine, Loratadine, and Pranlukast.

The non-limiting BCS Class IV drugs are selected from the group consisting of acetaminophen, folic acid, dexametasone, furosemide, meloxicam, metoclopramide, acetazolamide, furosemide, tobramycin, cefuroxmine, allopurinol, dapsone, doxycycline, paracetamol, metronidazole, nistatin, amoxicilin, aciclovir, trimetoprim Sulfate, erithromycin suspension, oxcarbazepine, modafinil, oxycodone, nalidixic acid, clorothiazide, tobramycin, cyclosporin, tacrolimus, paclitaxel, prostaglandines, prostaglandine E2, prostaglandine F2, prostaglandine E1, proteinase inhibitors, indinavire, nelfinavire, saquinavir, cytotoxics, doxorubicine, daunorubicine, epirubicine, idarubicine, zorubicine, mitoxantrone, amsacrine, vinblastine, vincristine, vindesine, dactiomycine, bleomycine, metallocenes, titanium metallocene dichloride, lipid-drug conjugates, diminazene stearate, diminazene oleate, chloroquine, mefloquine, primaquine, vancomycin, vecuronium, pentamidine, metronidazole, nimorazole, tinidazole, atovaquone, buparvaquone.

The above disclosed non-limiting BCS Class II and IV drugs can be a free acid, free base or neutral molecules, or in the form of an appropriate pharmaceutically acceptable salt, a pharmaceutically acceptable solvate, a pharmaceutically acceptable co-crystal, a pharmaceutically acceptable enantiomer, a pharmaceutically acceptable derivative, a pharmaceutically acceptable polymorph, pharmaceutically acceptable ester, pharmaceutically acceptable amide or a pharmaceutically acceptable prodrug thereof.

The pharmaceutical acceptable polymers and excipients of the invention are selected from the group consisting of: poly (acrylic acid), poly (ethylene oxide), poly (ethylene glycol), poly (vinyl pyrrolidone), poly (vinyl alcohol), polyacrylamide, poly (isopropyl acrylamide), poly (cyclopropyl methacrylamide), ethyl cellulose, carboxymethyl cellulose, hydroxyethyl cellulose, hydroxypropyl cellulose, hydroxypropyl methyl cellulose, cellulose acetate phthalate, alginic acid, carrageenan, chitosan, hyaluronic acid, pectinic acid, (lactide-co-glycolide) polymers, starch, sodium starch glycolate, polyurethane, silicones, polycarbonate, polychloroprene, polyisobutylene, polycyanoacrylate, poly (vinyl acetate), polystyrene, polypropylene, poly (vinyl chloride), polyethylene, poly (methyl methacrylate), poly (hydroxyethyl methacrylate), acrylic acid, butyl acrylate copolymer, 2-ethylhexyl acrylate and butyl acrylate copolymer, vinyl acetate and methyl acrylate copolymer, ethylene vinyl acetate and polyethylene terephthalate, ethylene vinyl acetate and polyethylene, polyethylene terephthalate, cellulose, methyl cellulose, hypromellose acetate succinate nf, hypromellose acetate succinate jp, hypromellose acetate succinate, hypromellose phthalate nf, hypromellose phthalate, low-substituted hydroxypropyl cellulose nf, low-substituted hydroxypropyl cellulose jp, low-substituted hydroxypropyl cellulose nf-low-substituted hydroxypropyl cellulose jp copolymer, hypromellose usp, hypromellose ep, hypromellose jp, hypromellose phthalate jp, hypromellose phthalate ep, hypromellose, hypromellose phthalate nf, hypromellose phthalate, low-substituted hydroxypropyl cellulose, methacrylates, cellulose acetate butyrate, polylactide-polyglycolide copolymers, polycaprolactone, polylactide, polyglycolide, polyvinylpyrrolidone-co-vinyl acetate, polyrethanes, polyvinyl caprolactam-polyvinyl acetate-polyethylene glicol graft copolymer, polyvinyl caprolactam, polyvinyl acetate, vinylpyrrolidone-vinyl acetate copolymer, vinylpyrrolidone, vinyl acetate, polyoxyethylene-polyoxypropylene copolymer, polyoxyethylene, polyoxypropylene, polyoxirane, povidone, polyethylene oxide, cellulose acetate, copovidone, povidone k12, povidone k17, povidone k25, povidone k30, povidone k90, hypromellose e5, hypromellose e4m, hypromellose k3, hypromellose k100, hypromellose k4m, hypromellose k100m, hypromellose phthalate hp-55, hypromellose phthalate hp-50, hypromellose acetate succinate 1 grade, hypromellose acetate succinate m grade, hypromellose acetate succinate h grade, cellulose acetate phthalate, cationic methacrylate, methacrylic acid copolymer type a, methacrylic acid copolymer type b, methacrylic acid copolymer type c, polymethylacrylates, polyvinyl alcohol, hydroxypropylmethylcellulose acetate succinate, ethyl acrylate, methyl methacrylate, trimethylammonioethyl methacrylate, ethyl acrylate-methyl methacrylate copolymer, butyl/methyl methacrylate-dimethylaminoethyl methacrylate copolymer, butyl/methyl methacrylate, dimethylaminoethyl methacrylate, methacrylic acid-ethyl acrylate copolymer, methacrylic acid, methacrylic acid-methyl methacrylate copolymer, methyl acrylate-methyl methacrylate-methacrylic acid copolymer, methyl acrylate, methyl methacrylate and diethylaminoethyl methacrylate copolymer, methyl methacrylate, diethylaminoethyl methacrylate, succinate, d-α-tocopheryl polyethylene glicol 100 succinate, d-α-tocopheryl polyethylene glicol, ethylene oxide, polypropylene oxide, polyvinyl alcohol-polyethylene glycol graft copolymer, methacrylic acid-ethyl acrylate copolymer, poloxamer, micronized poloxamer, polysorbate 20, polysorbate 40, polysorbate 60, polysorbate 80, ethylene glycol-vinyl alcohol graft copolymer, polydextrose nf, hydrogenated polydextrose nf, methacrylic acid copolymer, methacrylic acid and methyl methacrylate copolymer, methacrylic acid and ethyl acrylate copolymer, carbomer homopolymer, carbomer copolymer, carbomer interpolymer and others.

Other agents such as vitamin E, hydrogenated castor oil, ethoxylated glycerol, glyceryl triricinoleate, olive oil NF and others may be added.

Having generally described this invention, a further understanding can be obtained by reference to certain specific examples, which are provided herein for purposes of illustration only, and are not intended to be limiting unless otherwise specified.

### EXAMPLES

### Example 1A

### Materials and Methods

### Materials

Ketoprofen (KTO) powder (CAS 22071-15-4) was purchased from Research Pharmaceutical Ltd. (Bogota, Colombia).). Soluplus®, a polyvinylcaprolactam-polyvinyl acetate-polyethylene glycol graft co-polymer (57:30:13), Kollidon® VA64, a N-vinylpyrrolidone-vinyl-acetate (6:4); and Kollidon® SR, a polyvinyl-acetate-polyvinylpyrrolidone (providone) (8:2), were generously donated by BASF (SE, Ludwigshafen, Germany). The materials were thermally characterized by Thermal Gravimetric Analysis (TGA) and Differential Scanning calorimetry (DSC). For KTO the melting temperature (Tm) was about 96° C., its degradation temperature was about 185° C., and its heat of fusion was 109.5 J7g. For Soluplus® the glass transition temperature (Tg) was about 77° C., for Kollidon® VA64 the Tg was about 108° C.; and for Kollidon® SR the Tg was about 43° C.

The polymers used in the examples of the invention have the following physical properties as shown in the table below:

| | | Glass | |
|---|---|---|---|
| | | transition | Degradation |
| | Average molecular | temperature | temperature |
| Polymer | weight (g/mol) | (° C.) | (° C.) |
| Polyvinyl caprolactam- | 140,000 | ~77 | ~195 |
| polyvinyl acetate- | (measured by gel | | |
| polyethylene glycol | permeation | | |
| | chromatography) | | |
| Polyvinyl acetate- | 370,000 | ~42 | ~215 |
| polyvinylpyrrolidone | (measured by gel | | |
| | permeation | | |
| | chromatography) | | |
| Hypromellose acetate | 18,000 | ~121 | ~218 |
| succinate | (measured with | | |
| | SEC-MALLS) | | |

The organic solvent dimethylformamide (DMF) with a purity of 99.8% was purchased from PanReac, (Spain).

Film Casting

A bar applicator PA-5567 BYK-Gardner GmbH (Geretsried, Germany) was used to prepared the films. The applicator has a slot opening of 203.2 μm and generates films with thickness of about 110 μm. To generate the films dimethylformamide (DMF) was used as a solvent because it was one of the solvents that dissolved all the materials (KTO and polymeric excipients). The solubility of the materials and samples in the DMF was calculated with the DMF's density (0,948 g/ml) and a 5 ml volumetric flask. The ratio of concentration for the samples KTO-Soluplus® and KTO-Kollidon® VA64 was 1:4, for the samples KTO-Soluplus®/Kollidon® VA64-Kollidon® SR was 1:8. For the controls, only which consisted just one material, the concentration ratio in DMF was 1:1.

Solvent evaporation was performed by placing the film samples in a vacuum drying cabinet at 50° C. and 100 mbar for 3 hours. Sample preparation was based on a design of experiments, containing 20, 35 and 50% (wt %) of KTO loading dose in each polymeric excipient(s) combination: KTO-Soluplus®, KTO-Kollidon® VA 64, KTO-Soluplus®-Kollidon® SR, and KTO-Kollidon® VA64-Kollidon® SR. The samples were analyzed for film formation uniformity and recrystallization during 4 weeks.

### Optical Microscopy

Microscopy of samples was performed using a polarized optical microscope (Leitz Laborlux 12 Pool S. Germany) equipped with a Nikon D60 digital camera, 10.2 MB-pixel resolution. For some samples a gamma filter was used to better analyze the images. Six fields were analyzed in all samples and the numbers of crystals found in each field, as well as their size were followed for 4 weeks.

The stability of the sample was assessed based on the presence of an amorphous or crystalline phase or both. If no reversion of phases was observed during the test, a good stability was determined.

### Area and Particle Size Distribution

The particle size of the crystals was determined by microscopy methods using a calibrated ocular micrometer and DraftSight Version V1R4 (Dassault Systemes) software. The particle size distribution and all the measurements are based on the equivalent diameter of each crystal. The equivalent diameter is the diameter of a sphere having the same projected area as the particle. Average area of the few crystals were measured and reported in Table 1B.

### Preparation of Hot Melted Samples

A torque rheometer (Haake PolyLab QC, Thermo Scientific) was utilized in this study to mix the different materials. The Haake mixer has two counter-rotating Roller rotors (R600), generating mixing similar to twin screw extruders. The Haake mixer is generally used in the pharma research for small scale testing before scaling up to an extruder. The mixer gives sample's temperature in real time, and the torque is given by the resistance of the rotor's rotation caused by the presence of the sample. The Haake mixer is heated up electrically and cooled down by natural convection. One of the advantages of using a Haake mixer is that the residence time can be controlled and fixed for all the samples and can be controlled separately from the processing temperature and rotor rotation speed. Around 55 g of materials (Soluplus®, Kollidon® VA64, Kollidon® SR, KTO, KTO-Polymer excipient(s)) were mixed in the chamber with different KTO loading doses, 20, 35 and 50% wt. The composition of each sample, as well as their names, processing temperatures and rotor rotation speeds are given in Table 1A. Two different processing temperatures and two different rotor rotation speeds were applied to find the most suitable processing window. The different KTO loading doses, processing temperatures and rotor rotation speeds were chosen based on a design of experiments (DoE). After 360 seconds of mixing, the samples were taken out of the Haake mixer and cooled down to room temperature in open air by natural convection.

### Rheological Experiments

Rotational rheometry (TA Instruments AR 2000 ex, New Castle, Del.) was utilized to measure the steady viscosity of the samples (different concentrations of KTO in Soluplus®-Kollidon® SR) at a constant shear rate of 0.5/s using a parallel plat of 25 mm. The samples were loaded between the parallel plates at 120° C. and were sustained isothermally during the test.

### Thermal Gravimetric Analysis (TGA)

A TA Instruments Q500 thermogravimetric analyzer (TGA) was used to analyze the chemical and thermal stability of the Soluplus®, Kollidon® VA64, Kollidon® SR, Ketoprofen; and the samples at elevated temperatures. In a ramp heating test, 15 mg were placed in an aluminum pan and heated from room temperature to about 900° C. at a heating rate of 10° C./min. All the measurements were done following the standard ASTM E 1131.

### Differential Scanning Calorimetry (DSC)

A TA Instruments DSC Q500 equipped with a cooling system was used to carry out DSC measurements. The chamber was flushed with nitrogen at rate of 50 ml/min. Between 12 and 15 mg of each sample was placed in an aluminum pan with a lid and sealed. The samples were heated from ˜−20° C. to ˜110° C. at rate of 20° C./min. To analyze the amorphous transitions the samples were quenched during the cooling down cycle. The glass transition temperatures were measured in the second heating cycle of a heat-cool-heat loop. All the measurements were done following the standard ASTM D 3418.

The rest crystallinity was calculated based on the heat of fusion under the melting peak. The percentage was calculated as a ratio of the heat of fusion of the sample to the heat of fusion of pure KTO.

### In Vitro Dissolution Testing

For the dissolution study, capsules containing 200 mg of KTO were prepared from three samples: 50% KTO-50% Soluplus®, 50% KTO-25% Soluplus®-25% Kollidon® SR, and 35% KTO-33% Soluplus®-32% Kollidon® SR. An USP apparatus II (Hanson Research) was used in a paddle configuration method to perform USP 37. The samples were analyzed in 1000 ml of phosphate buffer with a pH of 7.4 and 0.05M. Temperature of the dissolution media was kept at 37±0.5° C. and the rotational speed was 50 rpm. Samples were taken at time intervals of 0.17, 0.33, 0.5, 1.0, 1.5, 2.0, 3.0, 4.0, 5.0, 6.0, 7.0, 8.0, 9.0, 10.0, 11.0, 12.0 hours. KTO quantification was performed by High-Performance Liquid Chromatography (HPLC) (Agilent Technologies 1260 Infinity) at 254 nm, with an injection volume of 20 μl. The percent release was calculated for all the capsules from the standard curves of KTO.

### Film Casting Procedure

Film casting is a technique where the API-polymeric excipient(s) pairs and possible combinations are solubilized with an organic solvent to generate films with a constant thickness. This technique is widely used to eliminate the API-polymer excipient that are not compatible and predicts the solubility of a certain API in a polymer matrix (Kolter et al., 2012; Reintjes 2011). The film stability and API recrystallization can be easily analyzed. It is a fast and suitable technique where the solid solution results in a clear and smooth film and API crystals can be recognized, as can amorphous precipitations, because they result in an opaque film.

### Binary Samples

The binary samples (KTO and one polymer excipient) were synthesized from KTO-Soluplus®, KTO-Kollidon® VA64 and KTO-Kollidon® SR with different KTO loading doses (20, 35 and 50%). Isolated crystals were observed, although they were difficult and hard to find. The crystals found in the samples were monitored for 4 weeks and data was collected after 24 h, 48 h, 1, 2 and 4 weeks. The average size of these crystals is reported in Table 2. Even though there are isolated crystals in the binary samples, the recrystallization of these samples is lower than in the pure KTO, where there was total recrystallization of the film. In fact, the crystals found in the KTO samples were bigger that the ones found in the binary samples (Table 2). Regarding the KTO-Kollidon® SR samples, no crystals were found in the films even though there were crystals in the Kollidon® SR film. All the films were clear and transparent during the 4 weeks. These findings suggest that Soluplus®, Kollidon® VA64 and Kollidon® SR are suitable polymeric excipients for KTO since KTO shows good solubility in these polymer excipients, and better stability than pure KTO.

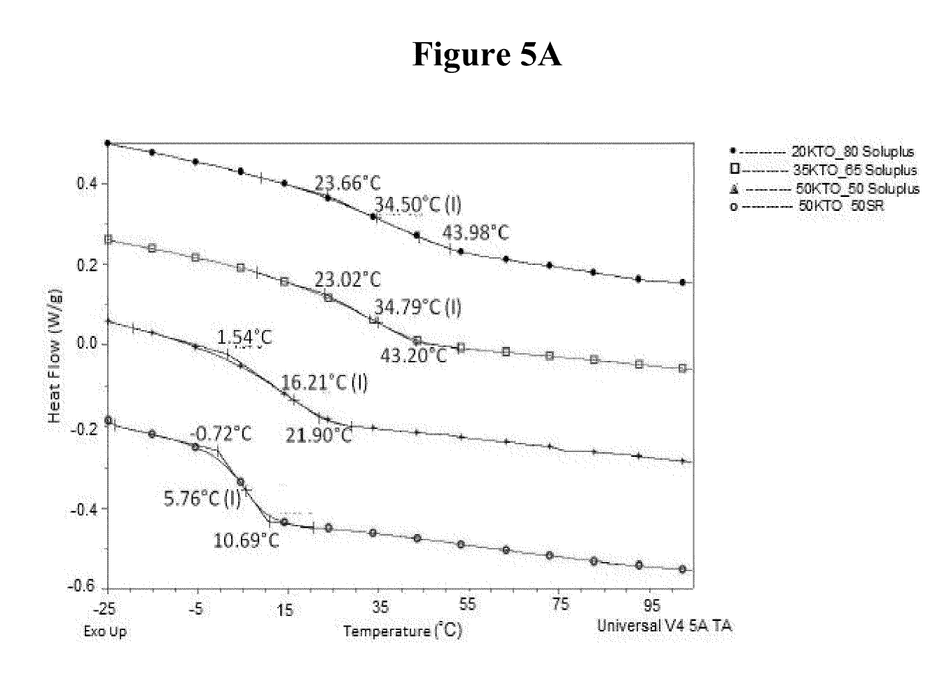

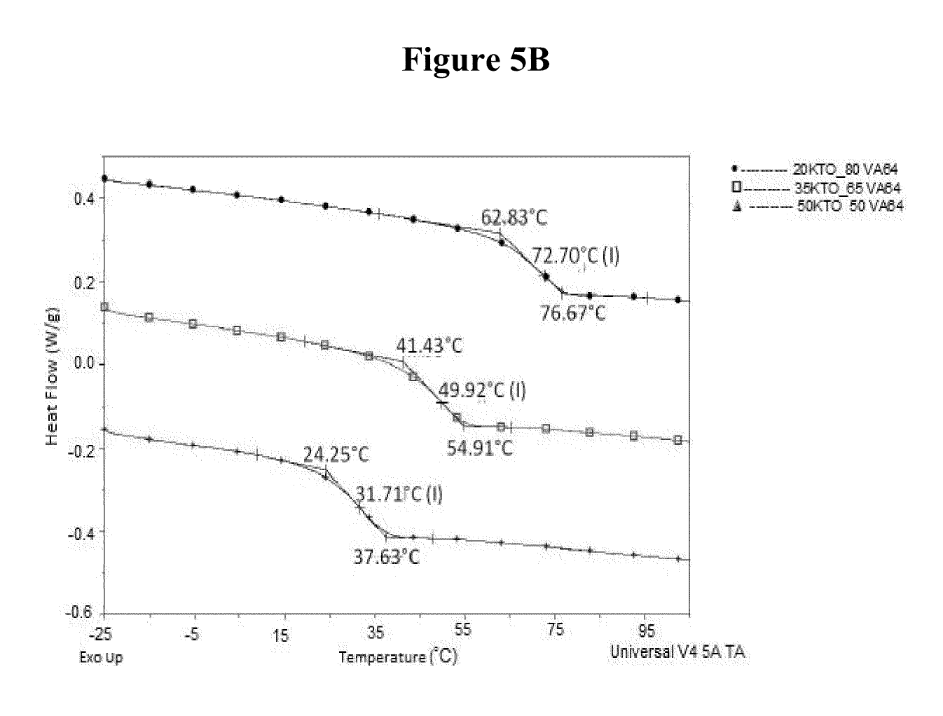

The first heating DSC thermogram showed rest crystallinity in the KTO-Soluplus® samples: 12.9% at 20% KTO, 13.6% at 35% KTO and 3.5% at 50% KTO. The samples KTO-Kollidon® VA64 and KTO-Kollidon® SR showed one glass transition temperature (Tg) in the first heating. In addition, the second heating DSC thermogram (FIGS. 5A and 5B) showed one glass transition temperature (Tg) for all the samples, indicating a single amorphous solid solution at 20, 35 and 50% KTO loading dose in Soluplus®, Kollidon® VA64 and Kollidon® SR. The films were kept at room temperature (23° C.) and the amorphous, single phase, solid solution remained during the 4 weeks assessment.

Soluplus® has a Tg around 77° C., Kollidon® VA64 around 108° C. and Kollidon® SR around 42° C.; and KTO has a melting point around 95° C. In FIG. 5B, it can be seen that as the loading dose of KTO increases, the Tg of the sample decreases, suggesting that the KTO behaves like a plasticizer. This finding is in agreement with the results reported by Crowley et al., in 2004, where samples containing more than 30% wt of KTO were not analyzed because they did not solidify after cooling.

The maximum loading dose prepared was 50% KTO (based on the DoE). At this concentration, the dissolution of KTO in the polymeric excipients was better than in the samples containing 20 or 35% API.

### Ternary Samples

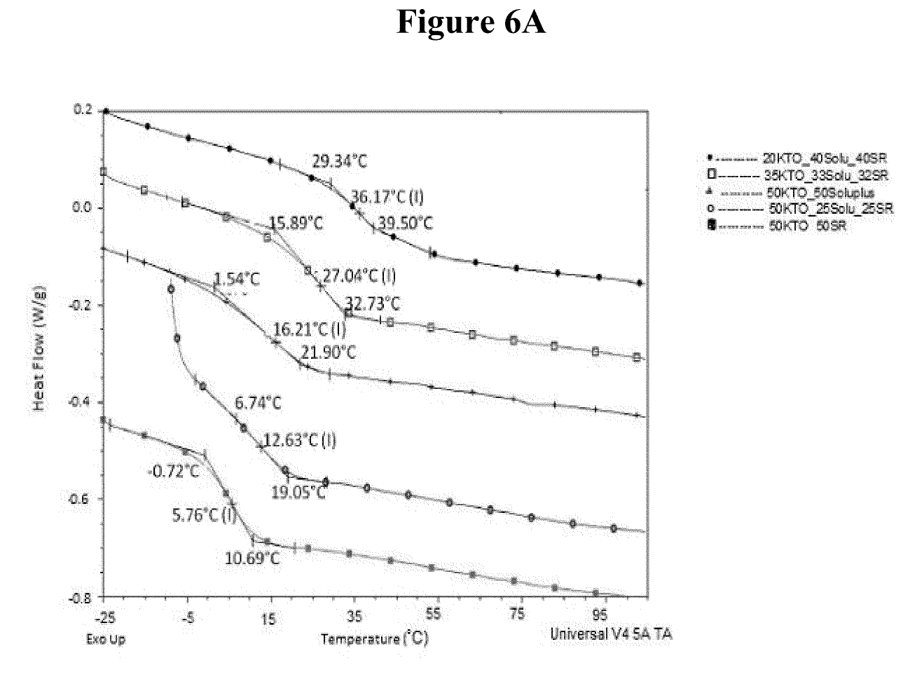

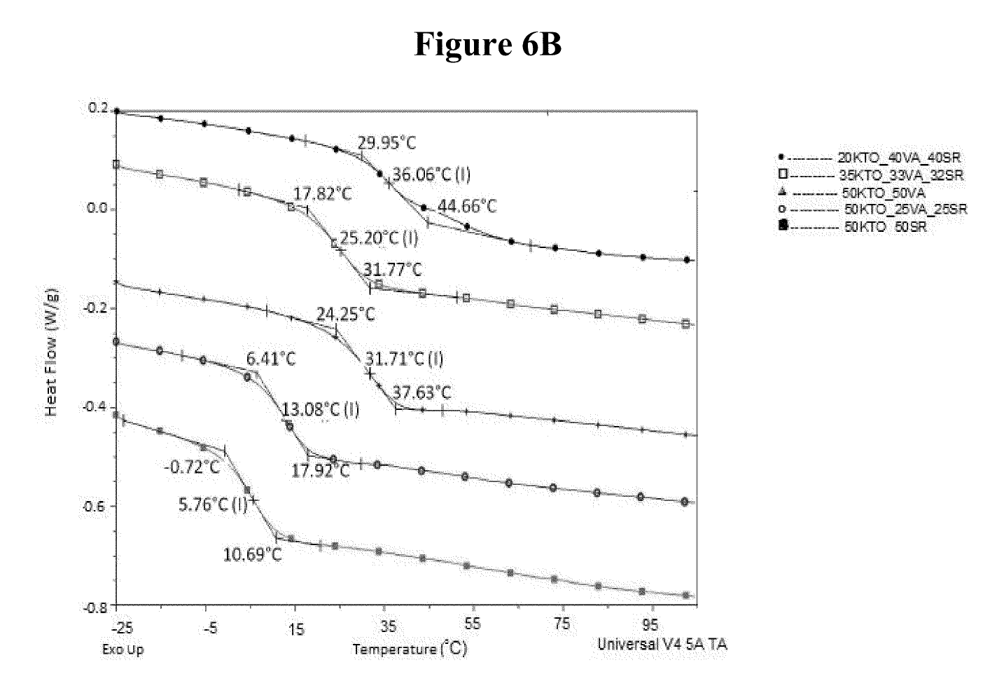

The ternary samples were prepared with different ratios of KTO, IR excipient (Soluplus® or Kollidon® VA64) and SR excipient (Kollidon® SR) 20:40:40, 35:33:32 and 50:25:25. Isolated crystals were found in the samples prepared with 20 and 35% KTO in Kollidon® VA64 and Kollidon® SR. However, no crystals were found in the samples containing 50% KTO in Kollidon® VA64 and Kollidon® SR not even in the samples of KTO, Soluplus® and Kollidon® SR. The area of these crystals is reported in Table 2. The few crystals found were smaller than the crystals found in pure KTO indicating good stability and interaction between the KTO and the two polymer excipients. It can be concluded that Soluplus® and Kollidon® VA64, in combination with Kollidon® SR are a good polymer excipient candidate for KTO. Nevertheless, the films were translucent, but since there was no crystallinity (FIGS. 6A and 6B)—crystals were present in the samples containing pure Kollidon® SR (Table 2)—the translucent appearance could be due to the hydrophilic-hydrophobic interactions between the IR and SR excipients.

The first heating from the DSC showed rest crystallinity in the samples containing a low loading of KTO. 7% rest crystallinity at 20% KTO 40% Soluplus® and 40% Kollidon® SR; 2.2% rest crystallinity at 35% KTO 33% Soluplus® 32% Kollidon® SR; 5.9% rest crystallinity at 20% KTO 40% Kollidon® VA64 40% Kollidon® SR; and 3.9% rest crystallinity at 35% KTO, 33% Kollidon® VA64 32% Kollidon® SR. The samples containing 50% KTO have only one transition, indicating the absence of rest crystallinity. The second heating from the DSC (FIGS. 6A and 6B) showed one Tg in all the ternary samples demonstrating that a single phase amorphous solid solution was achieved.

The films were kept at room temperature (23° C.) and analyzed for 4 weeks. The amorphous, single phase solid solution remained stable during the experiment, indicating good interaction between the polymer excipients and the API, and good stability.

### Hot Melt Mixing

Table 1 summarizes the samples prepared using the Haake mixer. It is reported in the literature that the processing temperature should be between the Tg of the polymer excipient and the melting point (Tm) of the API because passing the Tm of the API will degrade it. KTO has a Tm around 95° C. but it does not degrade until around 200° C., so the KTO melts before decomposition (Tita et al., 2013; Tita et al., 2011), This was confirmed by TGA, where the degradation temperature indicated was 185.79° C. Based on this information, a design of experiments was improved and two processing temperatures and rotor rotation speeds were selected. For samples with Soluplus® 90° C. and 120° C., and 70 and 100 rpm were chosen (Table 1), whereas for samples with Kollidon® VA64 120° C. and 150° C., and 70 and 100 rpm were chosen. However, the samples containing KTO and Kollidon® VA64 have very low viscosity and their processing was not possible, which is why they were no longer analyzed since they were not suitable for HME.

Samples prepared with KTO-Soluplus® and KTO-Soluplus®-Kollidon® SR did not completely solidify after cooling. They showed an elastic or chewy appearance at room temperature. These results were in agreement with Crowley et al., 2004, where they reported that samples with more than 30% KTO did not solidify after cooling. This elastic characteristic was evidenced stronger as the KTO loading dose increased.

### KTO-Soluplus®

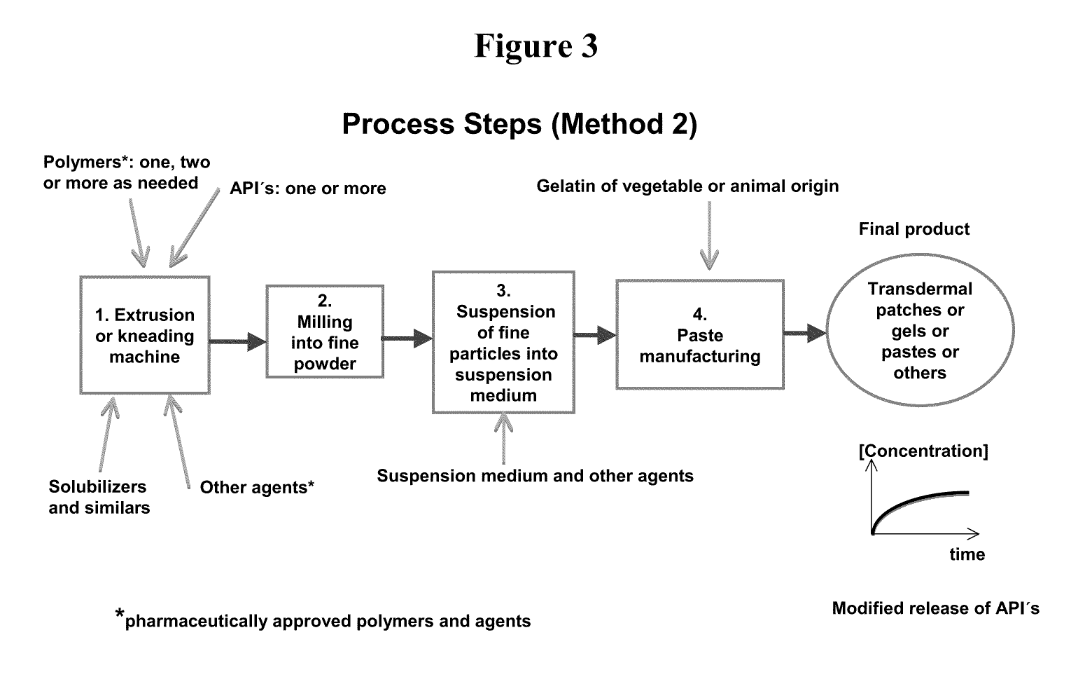

The DSC first heating at 20% KTO showed rest crystallinity at low processing temperature and low rotor rotation speed (4.2% rest crystallinity at 90° C. and 70 rpm), in contrast, with higher processing temperature and rotor rotation speed there was only one transition. The assessment is the same for 35% KTO, which showed 1.7% rest crystallinity at 90° C. and 70 rpm, and only one transition in the higher processing conditions. In the case of 50% KTO, single phase amorphous solid dispersion (only one Tg) was achieved in the first heating, confirming what was found with the film casting, at higher loading doses of API, the solubility of the API into the polymer excipients was better. The DSC second heating (FIG. 3) showed only one Tg indicating that a single phase amorphous solid solution was achieved at higher processing temperature (120°) and rotor rotation speed (70 rpm) for 20, 35 and 50% wt KTO. These results are in agreement with the results obtained by film casting.

### KTO-Soluplus®-Kollidon® SR

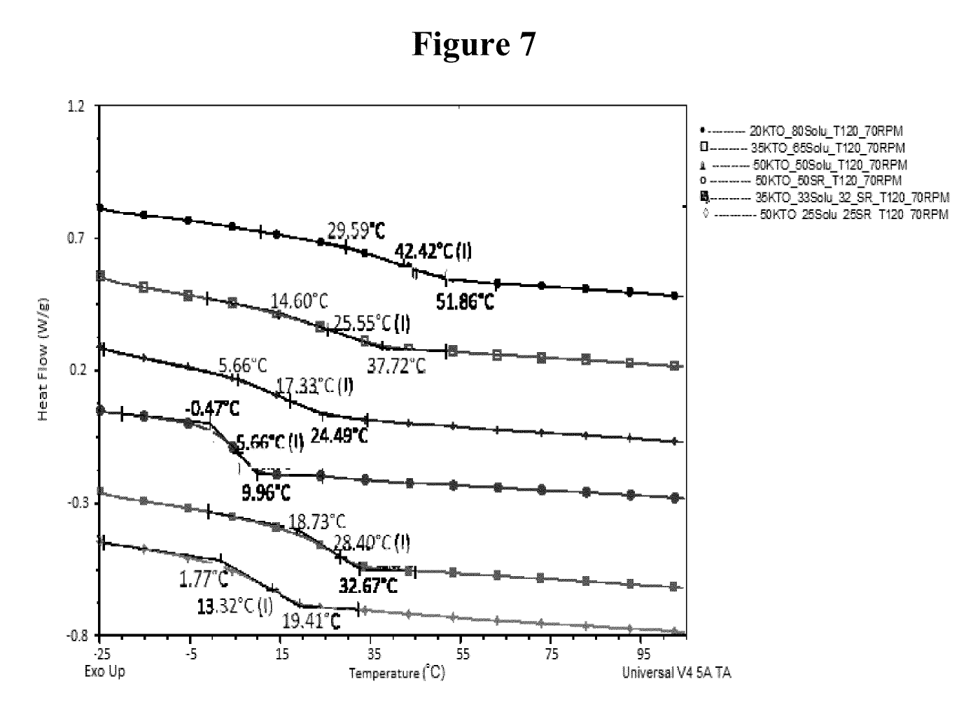

Samples with 20% KTO were no analyzed since film casting showed better results with higher KTO loading doses. The first heating in the DSC showed two transitions but not rest crystallinity, and the second heating (FIG. 7) showed only one Tg, indicating a single phase amorphous solid solution. In this stage, only the samples processed at 120° C. and 70 rpm were analyzed because they show the best behavior regarding the final torque. The results are in agreement with film casting.

The samples (Table 1) were analyzed by optical polarized microscopy and no recrystallization was found.

### Rheometric Measurements

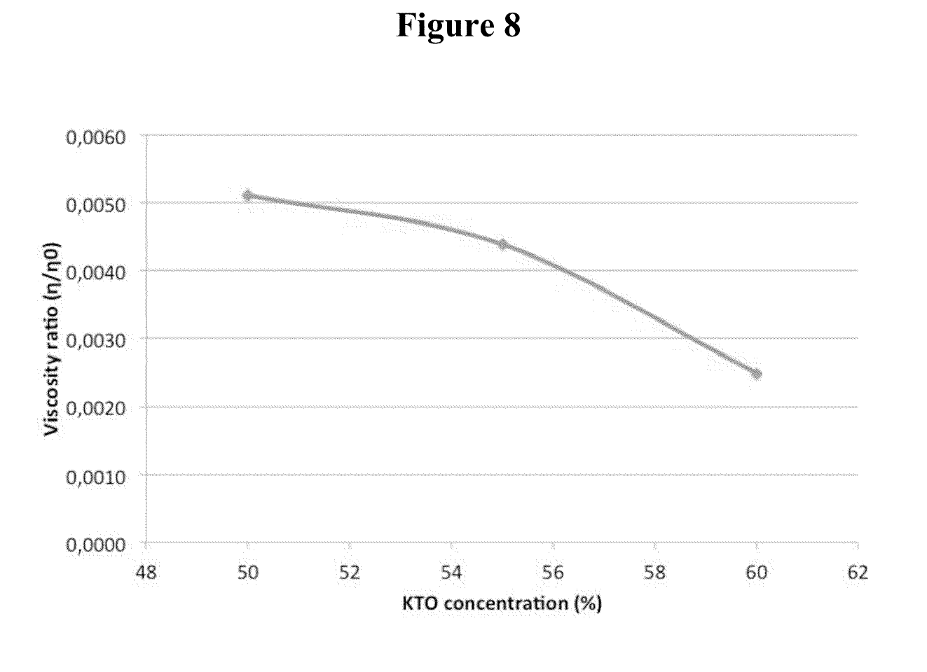

Rheometric measurements are very convenient to use because they can mimic the real HME process conditions, polymeric excipient characteristics and its mixtures with the API (Yang et al., 2011). More specifically, rheology can be applied to analyze the zero-shear viscosity of the polymeric excipient (η₀) and the API-polymeric excipient(s) mixture (η); the viscosity ratio (η/η₀) can be used to analyze the dissolution of the drug in the polymeric excipient(s) when increasing the drug concentration. The decrease in the ratio could indicate the disruption of the polymer structure due to the drug dissolution. On the other hand, the increase of the ratio could occur when the drug solubility has exceeded and there are undissolved solid drug particles in the excipient (Suwardie et al., 2011; Yang et al., 2011). FIG. 8 shows the decrease of viscosity ratio as increasing the concentration of KTO. This behavior corresponds to a good dissolution of KTO into Soluplus®-Kollidon® SR and the concentration of KTO could be increased. However, samples with more loading dose of KTO (more than 60% wt) were not analyzed because the viscosity was too low therefore it will be not possible to process these samples by HME. The decrease of viscosity ratio as increasing the KTO concentration strongly suggests that KTO acts as a plasticizer.

### In Vitro Drug Release

The dissolution of KTO (200 mg) in 1000 ml of 0.5M phosphate buffer (pH 7.4) containing different proportions of polymeric excipient(s) (Soluplus® or Soluplus®-Kollidon® SR) was conducted to analyze the influence of the IR and SR polymeric excipients in the KTO release. FIG. 5A shows the release profile of three different formulations: i) 50% KTO, 25% Soluplus® and 25% Kollidon® SR, ii) 50% KTO and 50% Soluplus®, and iii) 35% KTO, 33% Soluplus® and 32% Kollidon® SR.

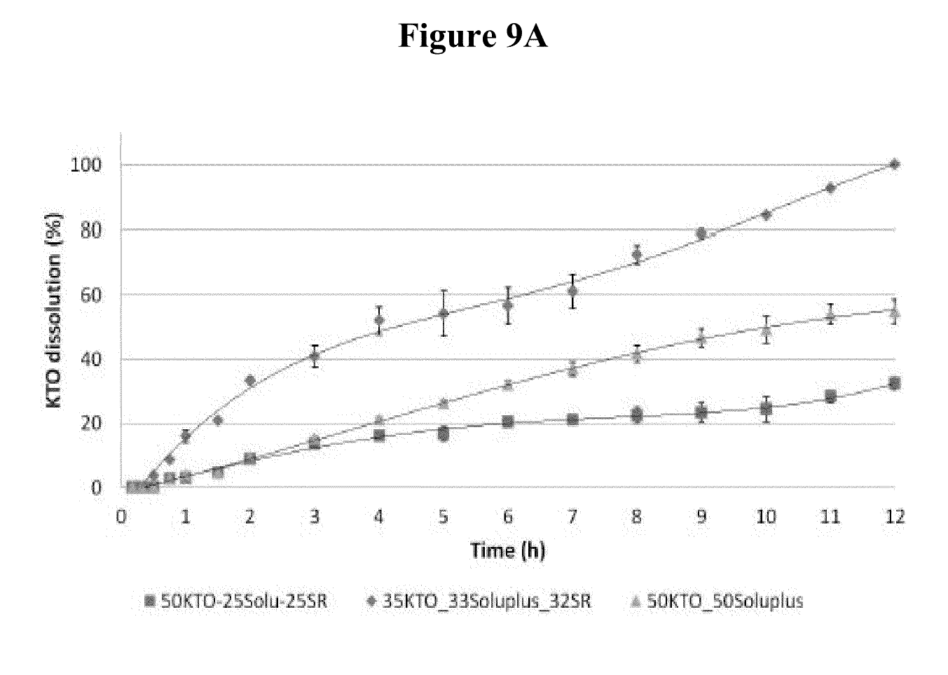

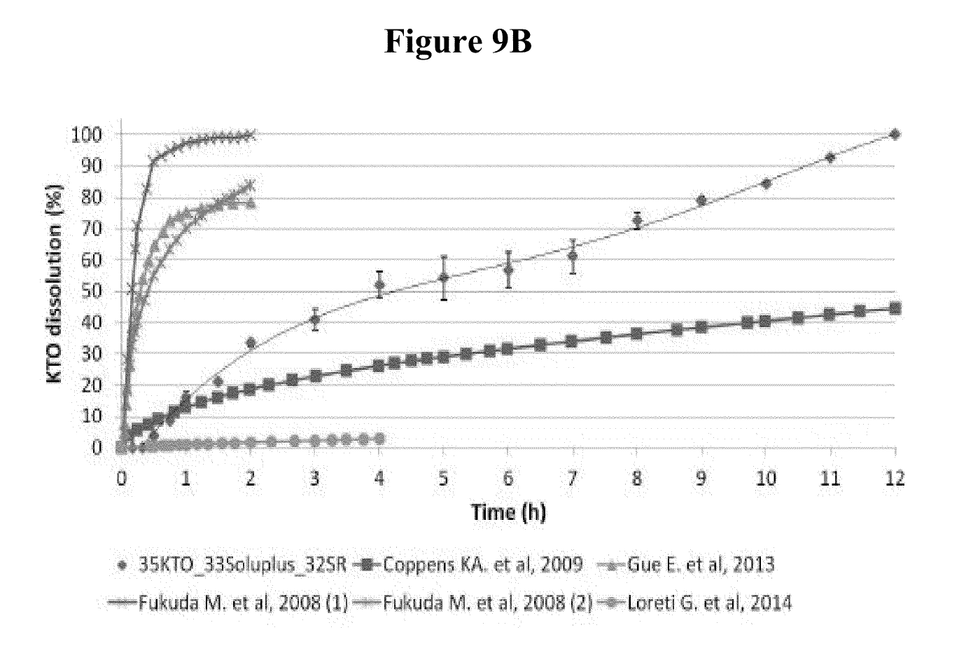

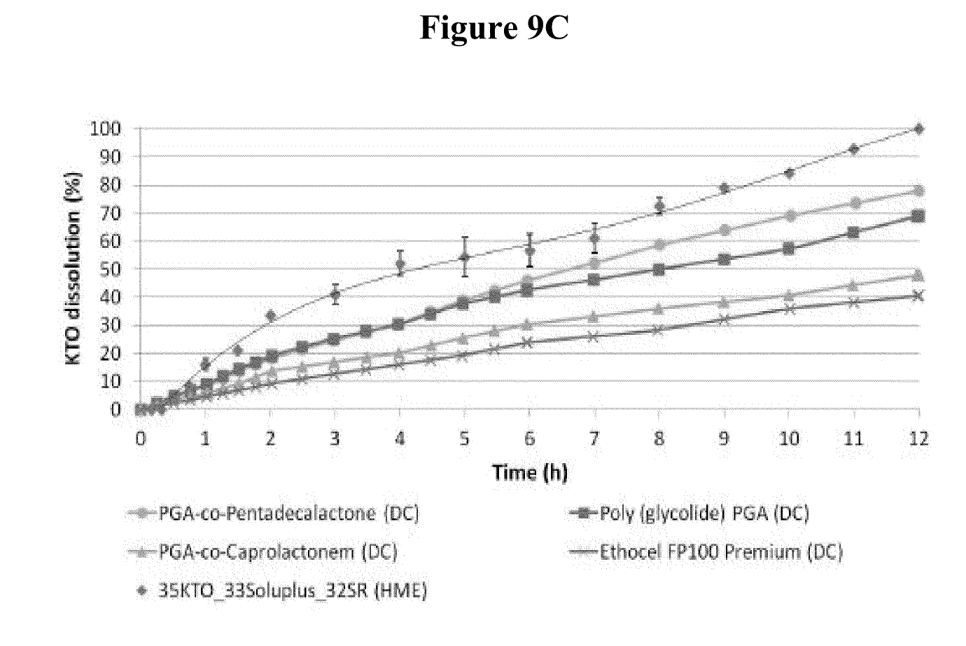

The formulation containing 50% KTO and 50% Soluplus® released 49.3% in 10 h and 54.9% in 12 h. This release profile does not meet the extended release requirement of minimum 90% release at 8 h (USP 37: protocol for extended release capsules—KTO). This behavior seems to be because the higher loading dose of KTO (50% wt) into the polymer excipients is not allowing the required release (Grund et al., 2014) and because Soluplus® acting as a pure polymer excipient interacts with the dissolution medium and the API cannot break through the polymer (FIG. 9A, triangle).

The formulation containing 50% KTO, 25% Soluplus® and 25% Kollidon® SR (50KTO-25Solu-25SR), showed 24.4% drug release in 10 h and 32.4% drug release in 12 h (FIG. 9A, square). This release profile does not meet the extended release requirement of minimum 90% release at 8 h (USP 37). Again, this behavior seems to be because the higher loading dose of KTO (50% wt) into the polymer excipients is not allowing the required release (Grund et al., 2014). The polymer mixture of Soluplus® and Kollidon® SR is not acting strong enough as solubility promoter. After 12 h, the test was discontinued, which is why it was not possible to achieve the intended 90% drug release. As expected, the release profile of 50KTO-25Solu-25SR is even more extended that the 50KTO-50Soluplus (FIG. 9A) owing to the fact that Kollidon® SR is a polymeric excipient design to create a sustained release matrix (Kollidon S R—Technical Information 2011).

The formulations containing 35% KTO and 65% polymeric excipients (33% Soluplus® and 32% Kollidon® SR) showed 84.3% drug release at 10 h and 100% release at 12 h (FIG. 9A, rhombi). The polymer excipients are acting as a promoter of solubility and allow the drug release. This release profile is considered as extended release by the USP 37 (specific for capsules containing 200 mg of KTO). The behavior is correlated with the results reported by Grund J. et al in 2014, where they report that samples containing around 60% polymer (v/v) have better release profile because the polymer concentration allows the drug release by percolation.

FIG. 9B shows a comparison of the extended release profile for the formulation containing 35% KTO, 33% Soluplus® and 32% Kollidon® SR (rhombi) with some releases of KTO found in the literature for formulations prepared by HME. Coopens K A. Et al, in 2009, reported a controlled release profile of formulation containing 20% KTO, 35% ETHOCEL Standard 10 Premium and 45% Polyox™ N10 (square in FIG. 9B). They accomplished around 45% drug release in 12 h. Gue E. et al, in 2013, reported an accelerated KTO release from polymeric matrices. The formulations were prepared with 30% KTO, 50% Eudragit™ E and 20% polyvinylpyrrolidone (PVP) (triangles in FIG. 9B). The dissolution test was performed for only 2 h and they accomplished around 80% drug release in that period of time. The samples contained 60 mg of KTO and the dissolutionmedium was 0.1 HCl. Fukuda M. et al, in 2008, reported the influence of sulfobutyl ether β-cyclodextrin and β-cyclodextrin on the dissolution of KTO from samples prepared by HME. They accomplished a KTO release of 100% in 2 h when the samples were prepared with 50% KTO and 50% sulfobutyl ether β-cyclodextrin (purple X in FIG. 5B); and a KTO release around 80% in 2 h when the samples were prepared with 50% KTO and 50% β-cyclodextrin (asterisks in FIG. 9B). The dissolution test was performed in 900 ml of 0.1M HCl at 37° C., 2.5 rpm and the samples contained 25 mg of KTO. Loreti G. et al, in 2014, reported the evaluation of the HME technique in the preparation of hydroxypropylcellulose (HPC) matrices for prolonged release. The formulations were prepared with 30% KTO and 70% HPC and the KTO release achieved was around 4% in 4 h (circles in FIG. 9B). The dissolution test was performed in 900 ml of deionized water at 37° C. and 50 rpm. It can be concluded that the release profile reported in this paper is more extended that the ones reported in the literature for KTO and samples prepared by HME.

The extended release profile of KTO obtained by HME was compared against direct compression (literature) in FIG. 9C. It could be seen that the KTO delivery was much faster by HME than by direct compression. The 100% extended release was achieved at 12 hours by HME. In the case of direct compression, extended release was not accomplished since the four samples showed a KTO delivery under 80% at 12 hours.

The first five hours of the dissolution test showed that the KTO delivery by HME is about 1.6 times faster than by direct compression due to the compaction involved in the last process. The samples prepared by direct compression contained different polymer excipients and KTO in a ratio of 10:2 (FIG. 9C).

In the instant invention, different characterization techniques have been applied to study KTO's solubility in Soluplus®, Kollidon® VA64 and Kollidon® SR in both, static (film casting) and dynamic conditions. Crystals were isolated and difficult to find, suggesting good stability of KTO in Soluplus®, Kollidon® VA64, Kollidon® SR and combinations of them. A single phase amorphous solid solution was achieved in all the different KTO-polymer excipient combinations at 20, 35 and 50% KTO loading dose (wt %) in the second heating DSC. In addition, it can be concluded that at higher KTO loading doses, the solubility of KTO in the polymer excipients is better, since the rest crystallinity of KTO decreases as the KTO loading dose increases. The same results were observed in the samples prepared by HME. The processing temperature and the rotor rotation speed play an important role in the solubility of the API in the polymer excipient(s). At lower processing temperature and lower rpm, there was rest crystallinity in the samples. Nevertheless, this rest crystallinity was eliminated at higher processing temperatures and rotor rotation speed, 120° C. and 70 rpm. Rheometric measurements suggested that the maximum KTO loading in Soluplus®-Kollidon® SR can be more than 60% (wt %). However, samples with more than 60% KTO were not analyzed since they were very elastic and their viscosity was too low making the samples not suitable for HME processing. Extended release was achieved with samples containing 35% KTO, 33% Soluplus® and 32% Kollidon® SR, releasing 100% KTO over 12 h. This release profile meets the extended release required by USP 37. HME is an advantageous technique that can be applied, in combination with IR and SR polymer excipients, for the manufacturing of extended release capsules containing 35% KTO, 33% Soluplus® and 32% Kollidon® SR. The recommended processing conditions for the Haake mixer are 120° C., 70 rpm and at least 120 seconds of residence time. Scale up is required to refine this processing window in a hot melt extruder.

TABLE 1A
Samples prepared by torque rheometer.

| | | Processing | |
|---|---|---|---|
| | Composition | temperatures | Rotor rotation |
| Name | (wt %) | (° C.) | speeds (rpm) |
| 20KTO-80Solu | 20% KTO, 80% | 90 and 120 | 70 and 100 |
| | Soluplus ® | | |
| 35KTO-65Solu | 35% KTO, 65% | 90 and 120 | 70 and 100 |
| | Soluplus ® | | |
| 50KTO-50Solu | 50% KTO, 50% | 90 and 120 | 70 and 100 |
| | Soluplus ® | | |
| 35KTO-33Solu- | 35% KTO, 33% | 90 and 120 | 70 and 100 |
| 32SR | Soluplus ®, | | |
| | 32% Kollidon ® SR | | |
| 50KTO-25Solu- | 50% KTO, 25% | 90 and 120 | 70 and 100 |
| 25SR | Soluplus ®, | | |
| | 25% Kollidon ® SR | | |
| 50KTO-50SR | 50% KTO, 50% | 90 and 120 | 70 and 100 |
| | Kollidon ® SR | | |

TABLE 1B
Average area (μm²) of the few found crystals in film
casting samples from film casting. Analyzed for 4 weeks.

| SAMPLE | 0.14 week | 0.28 week | 1 week | 2 weeks | 4 weeks |
|---|---|---|---|---|---|
| Ketoprofen | 104966.3 | 10534762.2 | 133207803.1 | 133207803.1 | 133207803.1 |
| Soluplus ® | — | — | — | — | — |
| Kollidon ® VA64 | — | — | — | — | — |
| Kollidon ® SR | 11008.7 | 11008.7 | 19863.5 | 19863.5 | 19863.5 |
| 20KTO-80Soluplus ® | — | — | 3884.5 | 17466.2 | 17466.2 |
| 35KTO-65Soluplus ® | 1592.7 | 1592.7 | 1592.7 | 1592.7 | 1592.7 |
| 50KTO-50Soluplus ® | 3247.7 | 3247.7 | 3247.7 | 3247.7 | 3247.7 |
| 20KTO-40Soluplus ®-40SR | — | — | — | — | — |
| 35KTO-33Soluplus ®-32SR | — | — | — | — | — |
| 50KTO-25Soluplus ®-25SR | — | — | — | — | — |
| 20KTO-80VA | — | 546.4 | 546.4 | 12206.7 | 12206.7 |
| 35KTO-65VA | 52408.2 | 52408.2 | 53718.7 | 53718.7 | 53718.7 |
| 50KTO-50VA | — | — | — | — | — |
| 20KTO-40VA-40SR | — | 3299.5 | 3299.5 | 3299.5 | 3299.5 |
| 35KTO-33VA-32SR | — | — | — | 1746.2 | 1746.2 |
| 50KTO-25VA-25SR | — | — | — | — | — |
| 50KTO-50SR | — | — | — | — | — |

### Example 1B

A controlled release dosage forms based on a nonsteroidal anti-inflammatory drug (NSAID) were prepared in accordance with the present invention and having the following composition shown in Table 1C.

TABLE 1C

| Ingredient | % w/w |
|---|---|
| Ketoprofen | 35-50% |
| Polyvinyl caprolactam-polyvinyl | 25-50% |
| acetate-polyethylene glycol | |
| Polyvinyl acetate-polyvinylpyrrolidone | 0-32% |

The above dosage forms were prepared by blending the ingredients for 6 minutes in a Haake lab mixer at 120° C. and 70 rpm. After the blending the samples were milled until reaching a particle size smaller than 180 μm. Then, the milled samples were encapsulated into hard gelatin capsules by adding the amount of sample needed to obtain 200 mg of ketoprofen in each capsule. The hard capsules were tested in simulated intestinal fluid (pH 7.4 phosphate buffer 0.05M) according to the procedure described in the United States Pharmacopeia 37 for 200 mg of Ketoprofen Extended-Release Capsules, using Apparatus II at 50 rpm. The dosage forms were found to have the following release profiles as shown in Table 2.

TABLE 2

| | 50% ketoprofen, 25% Polyvinyl | 35% ketoprofen, 33% Polyvinyl | 50% ketoprofen, |
|---|---|---|---|
| | caprolactam-polyvinyl acetate- | caprolactam-polyvinyl acetate- | 50% Polyvinyl |
| | polyethylene glycol, 25% | polyethylene glycol, 32% | caprolactam- |
| Time | Polyvinyl acetate- | Polyvinyl acetate- | polyvinyl acetate- |
| (h) | polyvinylpyrrolidone | polyvinylpyrrolidone | polyethylene glycol |
| 0.5 | 0.00 | 3.72 | 0.00 |
| 1 | 2.92 | 16.03 | 3.82 |
| 1.5 | 4.43 | 20.96 | 4.43 |
| 2 | 8.94 | 33.41 | 9.31 |
| 3 | 13.70 | 40.87 | 15.10 |
| 4 | 16.07 | 52.02 | 21.10 |
| 5 | 16.76 | 54.24 | 26.32 |
| 6 | 20.38 | 56.72 | 31.92 |
| 7 | 21.07 | 61.17 | 36.94 |
| 8 | 22.46 | 72.45 | 41.69 |
| 9 | 23.44 | 78.91 | 46.70 |
| 10 | 24.37 | 84.30 | 49.25 |
| 11 | 28.27 | 92.70 | 54.01 |
| 12 | 32.39 | 100.16 | 54.91 |

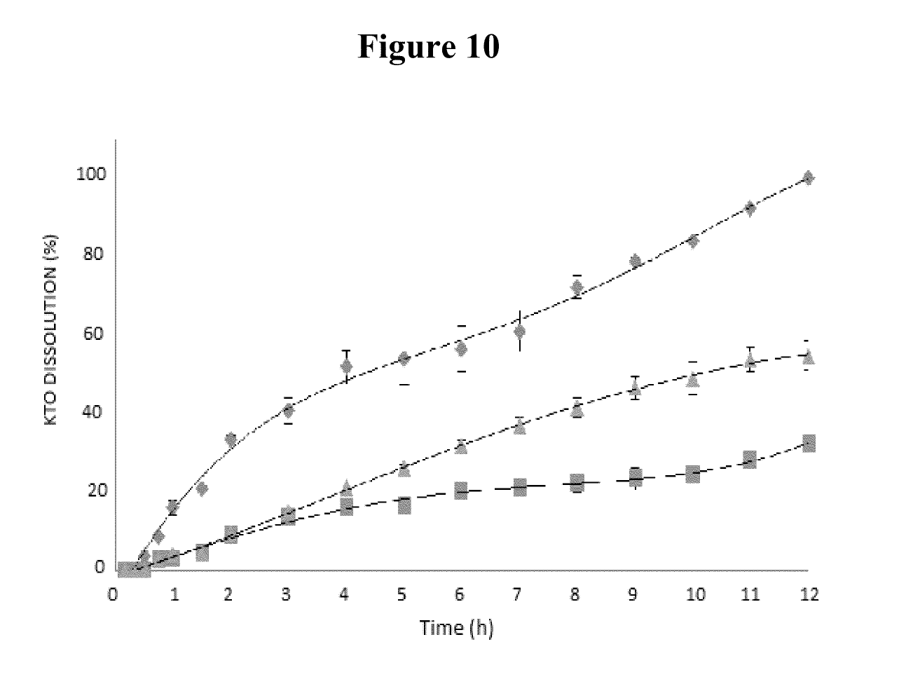

The release profiles in pH 7.4 phosphate buffer 0.05M of the controlled release dosage forms prepared in this example are shown in FIG. 10. In FIG. 10, the rhombus represent the sample with 35% Ketoprofen, 33% polyvinyl caprolactam-polyvinyl acetate-polyethylene glycol and 32% polyvinyl acetate-polyvinylpyrrolidone. The triangles represent the sample with 50% Ketoprofen and 50% polyvinyl caprolactam-polyvinyl acetate-polyethylene glycol. The squares represent sample with 50% Ketoprofen, 25% polyvinyl caprolactam-polyvinyl acetate-polyethylene glycol and 25% polyvinyl acetate-polyvinylpyrrolidone. Analyzed in a pH 7.4 phosphate buffer using Apparatus II at 50 rpm. As shown in FIG. 10, it is seen that it was possible to obtain an extended release profile of Ketoprofen after blending at 120° C. and 70 rpm during 6 minutes. The formulation with 35% API and 65% polymer excipients showed a better release profile achieving 100% ketoprofen in 12 hours.

### Example 2

A controlled release dosage forms based on a nonsteroidal anti-inflammatory drugs (NSAID) were prepared in accordance with the present invention and having the following composition shown in table 3.

TABLE 3

| Ingredient | % w/w |
|---|---|
| Ketoprofen | 35% |
| Polyvinyl caprolactam-polyvinyl | 33% |
| acetate-polyethylene glycol | |
| Polyvinyl acetate-polyvinylpyrrolidone | 32% |

The dosage forms were prepared by a twin-screw extruder at different processing conditions. The melt temperatures were setup at 115, 120 and 125° C., the screw rotation speeds were setup at 100 and 110 rpm, and the used extruder filling factors were 50, 60 and 70%. The dosage forms were the following as shown in table 4.

TABLE 4

| Dosage | Melt temperature | Screw rotation | Extruder Filling |
|---|---|---|---|
| form | (° C.) | speed (rpm) | factor (%) |
| 1 | 115 | 100 | 50 |
| 2 | 115 | 110 | 70 |
| 3 | 115 | 120 | 60 |
| 4 | 120 | 100 | 70 |
| 5 | 120 | 110 | 60 |
| 6 | 120 | 120 | 50 |
| 7 | 125 | 100 | 60 |
| 8 | 125 | 110 | 50 |
| 9 | 125 | 120 | 70 |

After extrusion the samples were milled until reaching a particle size smaller than 180 μm. Then, the milled samples were encapsulated into hard gelatin capsules by adding the amount of sample needed to obtain 150 mg of ketoprofen in each capsule. The hard capsules were tested in phosphate buffer, pH 6.8, according to the procedure described in the United States Pharmacopeia 37 for 150 mg of Ketoprofen Extended-Release Capsules, using Apparatus II at 50 rpm. The dosage forms were found to have the following release profiles shown in Table 5.

TABLE 5

| Time | Dosage form | | | | | | | | |
|---|---|---|---|---|---|---|---|---|---|
| (h) | 1 | 2 | 3 | 4 | 5 | 6 | 7 | 8 | 9 |
| 1 | 23.4 | 24.0 | 11.1 | 38.0 | 23.4 | 29.4 | 21.0 | 27.9 | 15.1 |
| 2 | 39.8 | 37.8 | 21.8 | 65.4 | 40.3 | 56.7 | 40.7 | 49.1 | 21.3 |
| 4 | 63.2 | 57.1 | 37.4 | 90.5 | 61.1 | 81.9 | 69.5 | 77.3 | 32.5 |
| 6 | 77.1 | 74.3 | 48.7 | 98.7 | 74.9 | 88.2 | 84.6 | 89.8 | 39.7 |
| 8 | 85.3 | 84.8 | 56.3 | 99.5 | 82.9 | 90.5 | 91.4 | 94.4 | 45.7 |
| 12 | 94.9 | 94.6 | 66.5 | 98.9 | 91.2 | 93.0 | 95.2 | 96.5 | 52.9 |
| 14 | 98.0 | 97.9 | 71.0 | — | 92.6 | 93.8 | 96.5 | 96.2 | 56.9 |
| 24 | 101.1 | 102.0 | 84.1 | — | 97.0 | 96.0 | 96.2 | 96.0 | 69.0 |

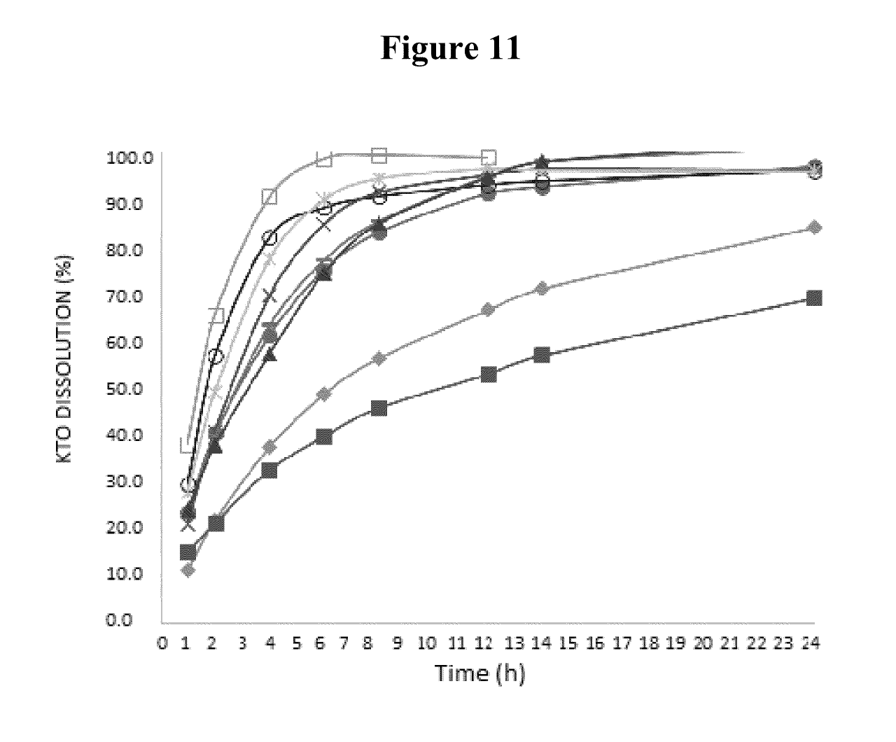

The release profiles in phosphate buffer, pH 6.8, of the controlled release dosage forms prepared in this example are shown in FIG. 11. As shown in FIG. 11, several release profile s of dosage forms were prepared by twin-screw extruder and containing 35% Ketoprofen, 32% polyvinyl caprolactam-polyvinyl acetate-polyethylene glycol and 32% polyvinyl acetate-polyvinylpyrrolidone under different processing conditions. Filled rhombus represent dosage form prepared at 115° C., 120 rpm and 60% of extruder filling factor. Filled circles represent dosage form prepared at 120° C., 110 rpm and 60% of extruder filling factor. X represent dosage form prepared at 125° C., 100 rpm and 60% of extruder filling factor.—represent dosage form prepared at 115° C., 100 rpm and 50% of extruder filling factor. Filled squares represent dosage form prepared at 125° C., 120 rpm and 70% of extruder filling factor. Hollow squares represent dosage form prepared at 120° C., 100 rpm and 70% of extruder filling factor. Hollow circles represent dosage form prepared at 120° C., 120 rpm and 50% of extruder filling factor. The asterisk represent dosage form prepared at 125° C., 110 rpm and 50% of extruder filling factor. Filled triangles represent dosage form prepared at 115° C., 110 rpm and 70% of extruder filling factor. Clearly, it was possible to scale up the good Haake lab mixer results to the extrusion process, achieving better release profiles in compliance with the Ketoprofen Extended-Release Capsule Official Monographs described in pharmacopeia USP 37. Finally, it was possible to obtain ketoprofen extended release profiles up to 12 hours by changing processing conditions of the extruder.

### Example 3

A controlled release dosage forms based on atypical antipsychotic active ingredient were prepared in accordance with the present invention and having the following composition shown in table 6.

TABLE 6

| Ingredient | % w/w |
|---|---|
| Quetiapine Fumarate | 35% |
| Polyvinyl caprolactam-polyvinyl | 33% |
| acetate-polyethylene glycol | |
| Hypromellose acetate succinate | 32% |

The dosage form was prepared by blending the ingredients for 6 minutes in a Haake lab mixer at 140° C. and 100 rpm. After the blending the dosage form was milled until reaching a particle size smaller than 180 μm. Then, the milled sample was encapsulated into hard gelatin capsules by adding the amount of sample needed to obtain 150 mg of Quetiapine base in each capsule. The hard capsules were tested in 900 mL of 0.05M citric acid and 0.09N NaOH (pH 4.8) for 5 hours, after the 5 hours the pH was adjusted to 6.6 by addition of 100 mL of 0.05M dibasic sodium phosphate and 0.46N NaOH, using Apparatus I at 200 rpm according to the procedure described in the FDA forum for Quetiapine Fumarate-extended release. The dosage form was found to have the following release profiles shown in table 7.

TABLE 7

| | 35% Quetiapine Fumarate, 32% polyvinyl caprolactam- |
|---|---|
| | polyvinyl acetate-polyethylene glycol, 32% hypromellose |
| Time (h) | acetate succinate |
| 1 | 22.6 |
| 2 | 33.3 |
| 4 | 47.5 |
| 6 | 60.2 |
| 8 | 69.2 |
| 10 | 75.2 |
| 12 | 80.5 |
| 14 | 84.1 |
| 24 | 91.8 |

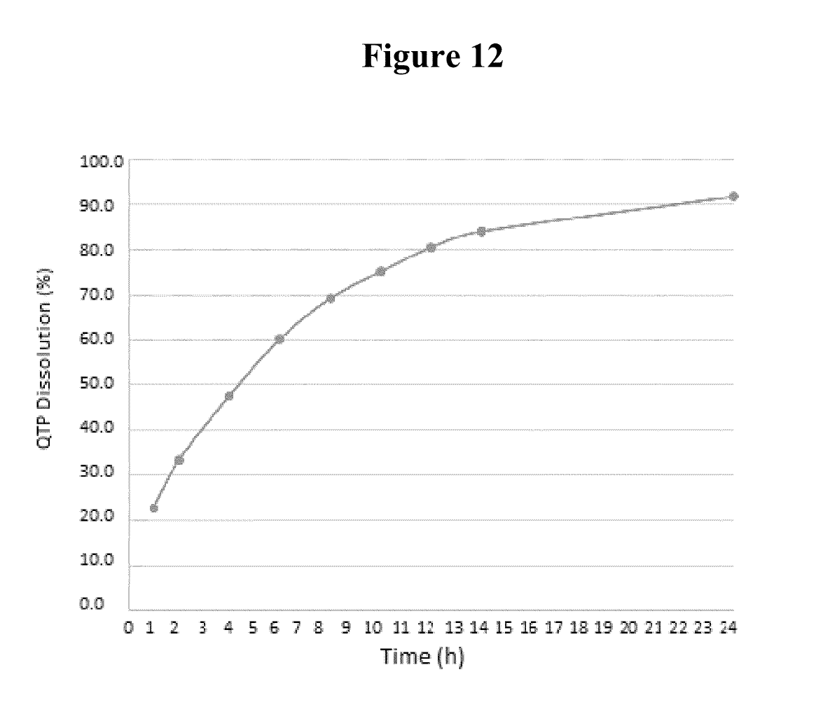

The release profiles in 0.05M citric acid and 0.09N NaOH (pH 4.8) for 5 hours, then the pH was adjusted to 6.6 by addition of 100 mL of 0.05M dibasic sodium phosphate and 0.46N NaOH, of the controlled release dosage forms prepared in this example are shown in FIG. 12. The release profiles in FIG. 12 correspond to blended dosage forms containing 35% Quetiapine Fumarate, 32% polyvinyl caprolactam-polyvinyl acetate-polyethylene glycol, and 32% hypromellose acetate succinate. The dosage form was analyzed in 0.05M citric acid and 0.09N NaOH (pH 4.8) for 5 hours, then the pH was adjusted to 6.6 by addition of 100 mL of 0.05M dibasic sodium phosphate and 0.46N NaOH. As shown in FIG. 12, it was possible to obtain an extended release profile of Quetiapine Fumarate after blending at 140° C. and 100 rpm during 6 minutes.

The formulation with 35% QTP and 65% polymer excipients showed an extended profile releasing 100% of Quetiapine Fumarate in 24 hours.

### Example 4

Controlled release dosage forms based on Fenofibrate were prepared in accordance with the present invention and having the following composition shown in table 8.

TABLE 8

| Ingredient | % w/w |
|---|---|
| Fenofibrate | 50% |
| Polyvinyl caprolactam-polyvinyl | 25% |
| acetate-polyethylene glycol | |
| Polyvinyl acetate-polyvinylpyrrolidone | 25% |

The dosage form was prepared by blending the ingredients for 6 minutes in a Haake lab mixer at 90° C., and 70 and 100 rpm. After the blending the dosage form was milled until reaching a particle size smaller than 180 μm. Then, the milled sample was encapsulated into hard gelatin capsules by adding the amount of sample needed to obtain 200 mg of Fenofibrate in each capsule. The hard capsules were tested in phosphate buffer with 2% Tween 80 and 0.1% pancreatin, pH 6.8, using Apparatus II at 75 rpm, according to the procedure described in the FDA forum for fenofibrate. The dosage form was found to have the following release profiles shown in table 9.

TABLE 9

| | 50% Fenofibrate, 25% polyvinyl | 50% Fenofibrate, 25% polyvinyl |
|---|---|---|
| | caprolactam-polyvinyl acetate- | caprolactam-polyvinyl acetate- |
| | polyethylene glycol, 25% | polyethylene glycol, 25% |
| | polyvinyl acetate- | polyvinyl acetate- |
| Time | polyvinylpyrrolidone. 90° C. | polyvinylpyrrolidone. 90° C. |
| (h) | and 70 rpm | and 100 rpm |
| 0.25 | 4.04 | 7.77 |
| 0.5 | 11.79 | 15.78 |
| 0.75 | 18.05 | 23.75 |
| 1 | 22.76 | 29.09 |
| 1.5 | 29.97 | 36.95 |
| 2 | 35.71 | 43.14 |
| 3 | 45.01 | 53.54 |
| 4 | 52.04 | 61.26 |
| 5 | 57.59 | 67.27 |
| 6 | 64.21 | 74.65 |
| 7 | 68.74 | 79.22 |
| 8 | 73.08 | 83.20 |
| 9 | 76.25 | 85.96 |
| 10 | 79.92 | 89.75 |
| 11 | 82.00 | 90.92 |
| 12 | 82.55 | 92.68 |
| 24 | 90.61 | 97.56 |

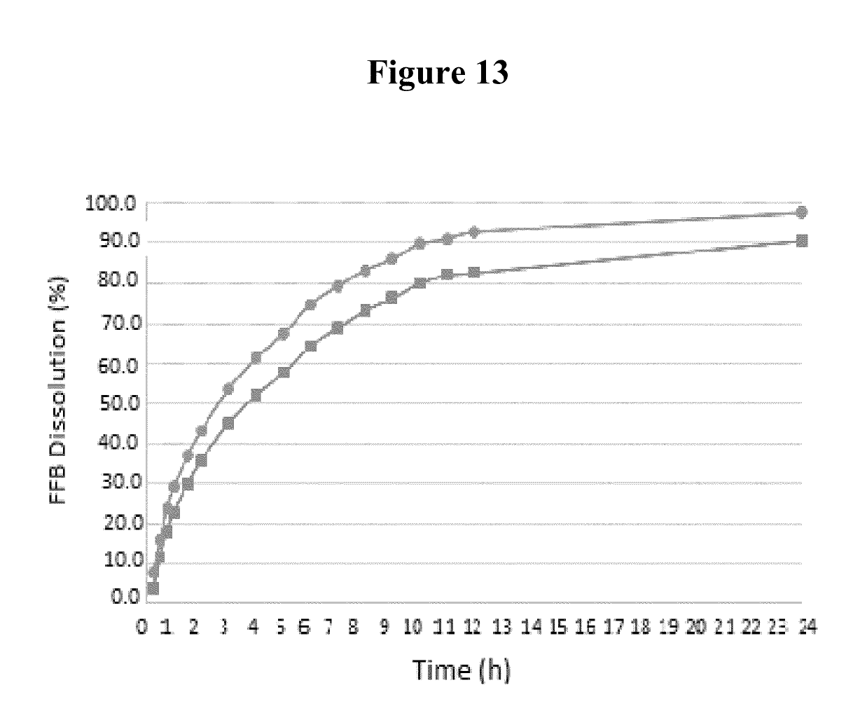

The release profiles in phosphate buffer with 2% Tween 80 and 0.1% pancreatin, pH 6.8, of the controlled release dosage forms prepared in this example are shown in FIG. 13. The release profiles shown in FIG. 13 are for blended dosage forms containing 50% Fenofibrate, 25% polyvinyl caprolactam-polyvinyl acetate-polyethylene glycol, 25% polyvinyl acetate-polyvinylpyrrolidone. Filled squares represent dosage form prepared at 90° C. and 70 rpm. Filled circles represent dosage form prepared at 90° C. and 100 rpm. FIG. 13 illustrates that it was possible to achieve an extended release profile of Fenofibrate after blending at 90° C., and 70 and 100 rpm during 6 minutes. The formulation with 50% FFB and 50% polymer excipients processed at 90° C. and 100 rpm showed a better release profile obtaining 100% of Fenofibrate in 24 hours.

### Example 5

A stability analysis was performed by Differential Scanning calorimetry (DSC) in samples prepared in accordance with the present invention and having the same composition shown in Table 3.

The samples of KTO-excipients were prepared by blending the ingredients during 6 minutes in a Haake lab mixer at 120° C. and 70 rpm. The stability analysis was done by Differential Scanning calorimetry (DSC) heating the sample from −20° C. up to 110° C. at a rate of 20° C./min. The DSC was carried out in samples right after preparation by Haake lab mixer and repeated again after 100 days. The absence of the KTO endothermal transition (i.e. melting: transition of crystalline to amorphous state) in the samples indicated that the drug remained in an amorphous state.

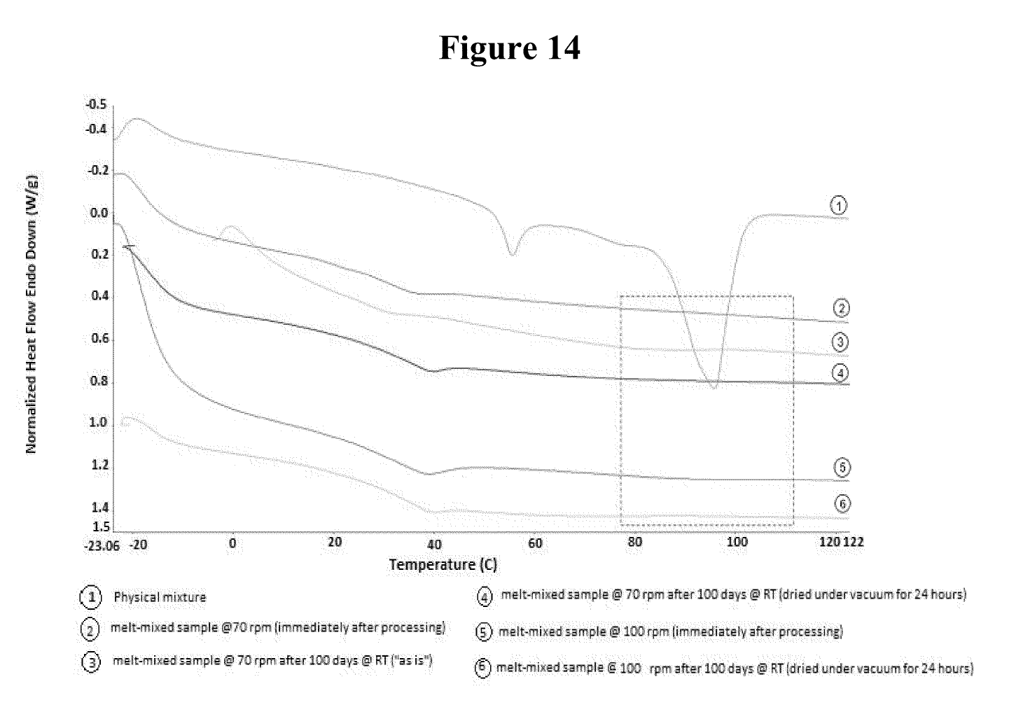

The stability assessment of KTO dosage forms by DSC is shown in FIG. 14. Stability assessment by Differential Scanning calorimetry were done on samples prepared by the Haake lab mixer. The analysis was performed immediately after processing and repeated 100 days later. In FIG. 14, \textcircled{1} represents the DSC thermal transition of physical mixture containing 35% Ketoprofen, 33% polyvinyl caprolactam-polyvinyl acetate-polyethylene glycol, and 32% polyvinyl acetate-polyvinyl-pyrrolidone. \textcircled{2} represents the DSC thermal transition of a sample containing 35% Ketoprofen, 33% polyvinyl caprolactam-polyvinyl acetate-polyethylene glycol, and 32% polyvinyl acetate-polyvinylpyrrolidone analyzed immediately after being processed by Haake lab mixer at 120° C. and 70 rpm. \textcircled{3} represents the DSC thermal transition of a sample containing 35% Ketoprofen, 33% polyvinyl caprolactam-polyvinyl acetate-polyethylene glycol, and 32% polyvinyl acetate-polyvinylpyrrolidone repeated 100 days after being processed by Haake lab mixer at 120° C. and 70 rpm. \textcircled{4} represents the DSC thermal transition of a sample containing 35% Ketoprofen, 33% polyvinyl caprolactam-polyvinyl acetate-polyethylene glycol, and 32% polyvinyl acetate-polyvinylpyrrolidone repeated 100 days after being processed by Haake lab mixer at 120° C. and 70 rpm and dried under vacuum for 24 hours. \textcircled{5} represents the DSC thermal transition of a sample containing 35% Ketoprofen, 33% polyvinyl caprolactam-polyvinyl acetate-polyethylene glycol, and 32% polyvinyl acetate-polyvinylpyrrolidone analyzed immediately after being processed by Haake lab mixer at 120° C. and 100 rpm. 0 represents the DSC thermal transition of a sample containing 35% Ketoprofen, 33% polyvinyl caprolactam-polyvinyl acetate-polyethylene glycol, and 32% polyvinyl acetate-polyvinylpyrrolidone repeated 100 days after being processed by Haake lab mixer at 120° C. and 100 rpm and dried under vacuum for 24 hours.

As shown in FIG. 14, the absence of a KTO endothermal transition (i.e. melting: transition of crystalline to amorphous state) in the samples analyzed immediately after preparation and repeated 100 days later indicated that the drug remained in an amorphous state. Therefore, the amorphous solid solution state was maintained.

### Example 6

### Encapsulating the Amorphous Solid Solution

In order to verify the feasibility of encapsulating (gelatin capsules) the amorphous solid solution, two sets of experiments were designed and performed. The first one was aimed at identifying a viscosity operating window for the capsule's fill. The second one was carried out to establish the technical feasibility and operational constraints of encapsulating a suspension carrying particles obtained from hot melt extrusion, exhibiting in their microstructure an amorphous solid solution. This section presents the design and conclusions of those two sets of experiments.

During the characterization stage, a factorial experiment 2⁴ with four central points and restrictions in the randomization was carried out, for a total of 20 experimental runs. The main objective of the experiment was to identify an operational region for the encapsulation process with viscosity as a design variable.

### 1. Factors

### 1.1. Qualitative:

Type of Gelatin: Ranked as high and low. (They correspond to typical bovine and porcine use as raw material for gel capsules manufacturing)

### 1.2. Quantitative:

Temperature of the segment: Low level (38° C.) C-High level (42° C.).
Viscosity: To run the tests, placebos substances were used, in order to modify viscosity levels.
Viscosity levels were: High (2500 cP), Medium (1750 cP), Low (1000 cp)
Machine Speed: We considered machine speed changes of 1 rpm, with speeds ranging from 2 rpm to 4 rpm.

| Factor | | Experimental levels | | | |
|---|---|---|---|---|---|
| Code | Factor | Low | M | High | Units |
| A: | Viscosity | 1000 | 1750 | 2500 | cP |
| B: | Temperature of the segment | 38 | 40 | 42 | ° C. |
| C: | Machine speed | 2.0 | 3.0 | 4.0 | rpm |
| D: | Type of Gelatin | I | — | II | |

### 1. Response Variables

| Response | | |
|---|---|---|
| Code | Description | Units |
| V1 | Dosing volume | ml |
| V2 | Relative Standar | % |
| | Deviation (RSD) for | |
| | Dosing Volume | |
| V3 | Upper Seal | % |
| V4 | Lower Seal | % |
| V5 | Hardness (Resistance | % |
| | to compression) | |
| V6 | RSD Hardness | % |

### 2. Analysis of the Response Variables Under Experimental Conditions

The experimental results are shown in the following table:

| A | B | C | D | V1 | V2 | V3 | V4 | V5 | V6 |
|---|---|---|---|---|---|---|---|---|---|
| 2500 | 38 | 4 | Tipo I | 1.02 | 0.27 | 14.75 | 14.75 | 3.85 | 11.15 |
| 1000 | 42 | 4 | Tipo II | 1.03 | 1.18 | 12.31 | 12.31 | 8.92 | 3.05 |
| 1750 | 40 | 3 | Tipo I | 1.02 | 7.19 | 14.75 | 14.75 | 4.54 | 7.34 |
| 2500 | 42 | 2 | Tipo II | 1.02 | 1.21 | 14.75 | 14.75 | 8.53 | 2.90 |
| 2500 | 42 | 4 | Tipo I | 1.20 | 1.92 | 16.00 | 16.00 | 3.91 | 8.45 |
| 1000 | 38 | 4 | Tipo I | 1.04 | 1.40 | 38.13 | 38.13 | 4.54 | 7.34 |
| 1000 | 38 | 2 | Tipo I | 1.03 | 1.66 | 23.38 | 23.38 | 4.51 | 4.80 |
| *1000 | 38 | 2 | Tipo II | 0.00 | 0.00 | 0.00 | 0.00 | 0.00 | 0.00 |
| 2500 | 38 | 2 | Tipo II | 1.02 | 0.57 | 17.22 | 17.22 | 9.33 | 2.08 |
| *2500 | 38 | 4 | Tipo II | 0.00 | 0.00 | 0.00 | 0.00 | 0.00 | 0.00 |
| *2500 | 42 | 4 | Tipo II | 0.00 | 0.00 | 0.00 | 0.00 | 0.00 | 0.00 |
| 1000 | 42 | 4 | Tipo I | 1.03 | 1.23 | 19.69 | 19.69 | 4.00 | 10.82 |
| 1750 | 40 | 3 | Tipo II | 1.03 | 0.42 | 14.75 | 14.75 | 9.25 | 2.77 |
| 2500 | 38 | 2 | Tipo I | 1.02 | 2.19 | 27.06 | 27.06 | 4.30 | 6.86 |
| 1000 | 42 | 2 | Tipo II | 1.02 | 0.56 | 32.00 | 32.00 | 8.94 | 3.56 |
| 1750 | 40 | 3 | Tipo II | 1.03 | 0.57 | 17.22 | 17.22 | 9.47 | 2.79 |
| 1000 | 42 | 2 | Tipo I | 1.02 | 7.78 | 23.38 | 23.38 | 4.19 | 5.01 |
| *1000 | 38 | 4 | Tipo II | 0.00 | 0.00 | 0.00 | 0.00 | 0.00 | 0.00 |
| 2500 | 42 | 2 | Tipo I | 1.01 | 1.00 | 23.38 | 23.38 | 4.02 | 8.21 |
| 1750 | 40 | 3 | Tipo I | 1.00 | 1.00 | 13.53 | 13.53 | 4.50 | 4.89 |

*The experimental levels which are shown with an asterisk did not exhibit a proper seal and it was not possible to measure any of the response variables.

### 1.1. Dosing Volume and RSD for Dosing Volume

V1 and V2 variables were analyzed to verify the statistical influence of experimental conditions, representing each of the variability factors thereof. Thus it was found that the type of gelatin and temperature factors are statistically significant on the behavior of the response variable, along with the interactions (type of gelatin—speed), (viscosity-type Gelatin—temperature) and (viscosity-type Gelatin—speed).

In the experimental area covered during the study with the gelatin type 1 (bovine), at speeds greater than 2 rpm, the dosing volume increased in the direction of rise of the viscosity and temperature. For speed equal to 2 rpm, the behavior of the response variable changed considerably. In the interval [38-40]° C. and [1000 to 2500] cP, the effect of viscosity is greater than the effect of temperature, showing two trends, a rise in the direction of [1000-1600] cP and a decrease of temperature in the intervals [1600-2500] cP.

For type 2 gelatin, the experimental study shows that at speeds of 2 rpm the dosing volume increases in the direction of rising temperature and falling viscosity. Moreover, at the speed of 2 rpm, the dosing volume increases in the direction of rise of the viscosity and temperature. The relative standard deviation is influenced by the type of gelatin and temperature factors. Its behavior exhibits a remarkable independency regardless of machine speed and viscosity.

### 1.2 Upper and Lower Seal

V3 and V4 variables were analyzed to verify the influence of design factors on the variability of the seals. In this case, it was found that the type of gelatin is a statistically significant factor in the response of these variables under analysis.

For type 1, bovine gelatin, a greater percentage of sealing at lower temperatures and in the direction of decrease in viscosity was observed. For gelatin type 2 (porcine) it was observed that it requires higher temperatures to achieve a better seal performance. This is partly attributed the behavior shown by non-sealed capsules where the response variables were give a zero value.

1.3 Addition of Particles from HME

A batch of extruded (by HME) was prepared and their samples milled to mesh size 80. It was verified that the microstructure exhibited an amorphous solid solution. A suspension was prepared with 40% solids using this particles and a pilot encapsulation process was run using a sub-region from the above experiment. The following observations were made:
Temperature increases diminished seal performance
There was no significant effect in response variables due to viscosity changes, as it was limited to the viscosity of the prepared suspension.
Encapsulation was observed for most testing conditions, with satisfactory seal performance.

The contents of all references cited in the instant specifications and all cited references in each of those references are incorporated in their entirety by reference herein as if those references were denoted in the text.

While the many embodiments of the invention have been disclosed above and include presently preferred embodiments, many other embodiments and variations are possible within the scope of the present disclosure and in the appended claims that follow. Accordingly, the details of the preferred embodiments and examples provided are not to be construed as limiting. It is to be understood that the terms used herein are merely descriptive rather than limiting and that various changes, numerous equivalents may be made without departing from the spirit or scope of the claimed invention.

### REFERENCES

Almeida A, Claeys B, Remon J P, Vervaet C. Hot-melt extrusion developments in the pharmaceutical industry. John Wiley & Sons Ltd. 1st ed. 2012.
Coppens K A, Hall M J, Koblinski B D, Larsen P S, Read M D, Shrestha U. Controlled release of poorly soluble drugs utilizing hot melt extrusion. The Dow Chemical Company 2009.
Crowley M M, Fredersdorf A, Schroeder B, Kucera S, Prodduturi S, Repka M A, McGinity J W. The influence of guaifenesin and ketoprofen on the properties of hot-melt extruded polyethylene oxide films. European J. of Pharm. Sci. 2004, 22:409-418
Dhirendra K, Lewis S, Udupa N, Atin K, Solid dispersions: A review. Pakistan J. of Pharm. Sci. 2009, 22:234-246.
Dixit M, Kulkarni P K, Vaghela R S. Effect of different crystallization techniques on the dissolution behavior of ketoprofen. Trop J Pharm Res, 2013, 12(3):317-322.
Djuris J, Nikolakakis I, Ibric S, Djuric Z, Kachrimanis K. Preparation of carbamazepine-Soluplus® solid dispersions by hot-melt extrusion, and prediction of drug-polymer miscibility by thermodynamic model fitting. European J. of Pharm. and Biopharm. 2013, 84:228-237.
Fukudaa M, Miller D A, Peppas N A, McGinity J W. Influence of sulfobutyl ether β-cyclodextrin (Captisol®) on the dissolution properties of a poorly soluble drug from extrudates prepared by hot-melt extrusion. Int. J. of Pharm. 2008, 350:188-196.
Grund J, Koerber M, Walther M, Bodmeiser R. The effect of polymer properties on direct compression and drug release from water-insoluble controlled release matrix tablets. Int. J. of Pharm. 2014, 469:94-101.
Gue E, Willart J F, Muschert S, Danede F, Delcourt E, Descamps M, Siepmann J. Accelerated ketoprofen release from polymeric matrices: Importance of the homogeneity/heterogeneity of excipient distribution. Int. J. of Pharm. 2013, 457:298-307 Jan S U, Khan G M, Khan H, Asim-ur-Rehman, Khan K A, Shag S U, Shah K U, Badshah A,
Hussain I. Release pattern of three new polymers in Ketoprofen controlled-release tablets. African J. of Pharm. and Pharmacol. 2012, 6(9):601-607. 10.5897/AJPP11.604.
Kalivoda A, Fischbach M, Kleinebudde P. Application of mixtures of polymeric carriers for dissolution enhancement of fenofibrate using hot-melt extrusion. Int. J. of Pharm. 2012, 429:58-68.
Kollidon S R, polyvinyl acetate and povidone based matrix sustained release excipient—Technical Information. BASF The chemical Company, Pharma ingredients & Services. 2011
Kolter K, Karl M, Gryczke A. Hot-melt extrusion with BASF Pharma polymers—Extrusion compendium 2nd revised and enlarge edition. Ludwigshafen, Germany. BASF The Chemical Company. 2012. ISBN 978-3-00-039415-7.
Loreti G, Maroni A, Del Curto M D, Melocchi A, Gazzaniga A, Zema L. Evaluation of hot-melt extrusion technique in the preparation of HPC matrices for prolonged release. European J. of Pharm. Sci. 2014, 52:77-85.
Maschke A, Klumpp U, Guntherberg N, Kolter K. Effect of Preparation Method on Release Behavior of Kollidon S R Tablets Hot Melt Extrusion versus Direct Compression.BASF the Chemical Company. Ludwigshafen, Germany. 2011.
Niu X, Wan L, Hou Z, Wang T, Sun C, Sun J, Zhao P, Jiang T, Wang S. Mesoporous carbon as a novel drug carrier of fenofibrate for enhancement of the dissolution and oral bioavailability. Int. J. of Pharm. 2013, 452:382-389.
Prodduturi S, Urman K, Otaigbe J, Repka M. Stabilization of hot-melt extrusion formulations containing solid solutions using polymer blends. AAPS Pharm. Sci. Tech. 2007, 8(2):E152-E161.
Reintjes T. Solubility Enhancement with BASF Pharma Polymers—Solubilizer Compedium. Ludwigshafen, Germany. BASF The Chemical Company. 2011.
Sarode A L, Sandhu H, Shah N, Malick W, Zia H. Hot melt extrusion (HME) for amorphous solid dispersions: Predictive tools for processing and impact of drug-polymer interactions on supersaturation. European J. of Pharm. Sci. 2013, 48:371-384.
Shah S, Maddineni S, Lu J, Repka M A. Melt extrusion with poorly soluble drugs. Int. J. of Pharm. 2013, 453:233-252.
Shoin I E, Kulinich J I, Ramenskaya G V, Abrahamsson B, Kop S, Langguth P, Polli J E, Shah V P, Groot D W, Barends D M, Dressman J B. Biowaiver Monographs for Immediate-Release Solid Oral Dosage Forms: Ketoprofen. J. of Pharm. Sci. 2012, 101(10):3593-3603.
Suwardie H, Wang P, Todd D B, Panchal V, Yang M, Gogos C G. Rheological study of the mixture of acetaminophen and polyethylene oxide for hot-melt extrusion application. European J. of Pharm. and Biopharm. 2011, 78:506-512.
Tita D, Fulias A. Tita Bogdan. Thermal stabilidty of ketoprofen-active substance and tablets. Part 1: kinetic study of the active substance under non-isothermal conditions. J. Therm. Anal. calorim. 2011, 105:501-508.
Tita D, Fulias A. Tita Bogdan. Thermal stabilidty of ketoprofen. Part 2: kinetic study of the active substance under isothermal conditions. J. Therm. Anal. calorim. 2013, 111:1979-1985.
Vueba M, Batista de cavalho L A E, Veiga F, Soussa J J, Pina M E. Influence of cellulose ether polymer on ketoprofen release from hydrophilic matrix tablets. European J. of Pharm. and Biofarm. 2004, 58:51-59.
Vueba M L, Pina M E, Veiga F, Sousa J J, Batista de cavalho LAE. Conformational study of Ketoprofen by combined DFT calculations and Raman spectroscopy. Int. J. of Pharm. 2006, 307:56-65.
Yadav S P, Kumar V, Singh U P, Bhat H R, Mazumder B. Physicochemical characterization andin vitro dissolution studies of solid dispersions of ketoprofen with PVP K30 andD-mannitol. Saudi Pharm. J. 2013, 21:77-84.
Yang M, Wang P, Huang C Y, Ku M S, Liu H, Gogos C. Solid dispersion of acetaminophen and poly(ethylene oxide) prepared by hot-melt mixing. Int. J. of Pharm. 2010, 395:53-61.
Yang M, Wang P, Suwardie H, Gogos C. Determination of acetaminophen's solubility in poly(ethylene oxide) by rheological, thermal and microscopic methods. Int. J. of Pharm. 2011, 403:83-89.

What is claimed is:

1. A method for making a dosage form comprising:
(a) preparing by melt extrusion or kneading an amorphous solid solution of one or more active ingredients; and
(b) placing said amorphous solid solution of said active ingredient in a suitable dosage form.

2. The method of claim 1, wherein said amorphous solid solution is a single phase solid solution.

3. The method of claim 1, where said active ingredient is selected from the group consisting of Class II and Class IV biopharmaceutical classes

4. The method of claim 1, where said amorphous solid solution includes one or more pharmaceutically or nutraceutical or food acceptable polymers and mixtures thereof

5. The method of claim 1, where said method does not exceed the degradation or decomposition temperature of said active ingredient.

6. The method of claim 5, where said method does exceed the melting point of the active ingredient when the decomposition or degradation temperature is near or above the melting point of said active ingredient.

7. A method according to claim 1, where said amorphous solid solution is milled subsequent to extrusion or kneading process

8. A method according to claim 7, where the milled amorphous solid solution is suspended in a suitable fluid

9. A method according to claim 8, where said fluid is a pharmaceutically or nutraceutical or food acceptable fluid

10. A method according to claim 8, where said milled amorphous solid solution is processed into a tablet

11. A method according to claim 8, where said milled amorphous solid solution is processed into pellets or microgranules

12. A method of claim 1, where said dosage form is a softgel capsule.

13. A method of claim 1, where said dosage form is a hard capsule.

14. A method of claim 1, wherein said dosage form is a transdermal dosage form.

15. A method of claim 1, wherein said active ingredient is a biologically active pharmaceutical.

16. A method of claim 1, wherein said active ingredient is a nutraceutical or dietary supplement.

17. A method of claim 1, wherein said active ingredient is incorporated in a functional food.

18. A method of claim 14, wherein said transdermal dosage form is in the form of a gel or paste.

19. A method of claim 14, wherein said transdermal dosage form is in the form of a transdermal patch

20. A method for enhancing the bioavailability of a biologically active ingredient in a mammal, which method comprises administering to said mammal an effective amount of an amorphous solid solution of said biologically active ingredient.

21. The method of claim 20, wherein said amorphous solid solution is a single phase solid solution.

22. A milled amorphous solid solution of a biologically active ingredient.

23. The milled amorphous solid solution of claim 22, wherein said amorphous solid solution is a single phase solid solution.

24. A dosage form incorporating an amorphous solid solution of at least one biologically active ingredient.

25. The dosage form of claim 24, wherein said amorphous solid solution is a single phase solid solution.

26. The dosage form of claim 25, wherein said biologically active ingredient belongs to BCS class II and/or IV.

27. A method for making a dosage form comprising:
(a) preparing by melt extrusion or kneading an amorphous solid solution of at least one active pharmaceutical ingredient (API) which belongs to BCS class II and/or IV; and
(b) placing said amorphous solid solution of said active ingredient in a suitable dosage form.

28. The method of claim 27, wherein said amorphous solid solution is a single phase solid solution.

29. The method of claim 27, wherein said BCS Class II drugs are selected from the group consisting of Albendazole, Acyclovir, Azithromycin, Cefdinir, Cefuroxime axetil, Chloroquine, Clarithromycin, Clofazimine, Diloxanide, Efavirenz, Fluconazole, Griseofulvin, Indinavir, Itraconazole, Ketoconalzole, Lopinavir, Mebendazole, Nelfinavir, Nevirapine, Niclosamide, Praziquantel, Pyrantel, Pyrimethamine, Quinine, Ritonavir, Bicalutamide, Cyproterone, Gefitinib, Imatinib, Tamoxifen, Cyclosporine, Mycophenolate mofetil, Tacrolimus, Acetazolamide, Atorvastatin, Benidipine, Candesartan cilexetil, Carvedilol, Cilostazol, Clopidogrel, Ethylicosapentate, Ezetimibe, Fenofibrate, Irbesartan, Manidipine, Nifedipine, Nisoldipine, Simvastatin, Spironolactone, Telmisartan, Ticlopidine, Valsartan, Verapamil, Warfarin, Acetaminophen, Amisulpride, Aripiprazole, Carbamazepine, Celecoxib, Chlorpromazine, Clozapine, Diazepam, Diclofenac, Flurbiprofen, Haloperidol, Ibuprofen, Ketoprofen, Lamotrigine, Levodopa, Lorazepam, Meloxicam, Metaxalone, Methylphenidate, Metoclopramide, Nicergoline, Naproxen, Olanzapine, Oxcarbazepine, Phenyloin, Quetiapine, Risperidone, Rofecoxib, Valproic acid, Isotretinoin, Dexamethasone, Danazol, Epalrestat, Gliclazide, Glimepiride, Glipizide, Glyburide (glibenclamide), levothyroxine sodium, Medroxyprogesterone, Pioglitazone, Raloxifene, Mosapride, Orlistat, Cisapride, Rebamipide, Sulfasalazine, Teprenone, Ursodeoxycholic Acid, Ebastine, Hydroxyzine, Loratadine, and Pranlukast.

30. The method of claim 27, wherein said BCS Class IV drugs are selected from the group consisting of acetaminophen, folic acid, dexametasone, furosemide, meloxicam, metoclopramide, acetazolamide, furosemide, tobramycin, cefuroxmine, allopurinol, dapsone, doxycycline, paracetamol, metronidazole, nistatin, amoxicilin, aciclovir, trimetoprim Sulfate, erithromycin suspension, oxcarbazepine, modafinil, oxycodone, nalidixic acid, clorothiazide, tobramycin, cyclosporin, tacrolimus, paclitaxel, prostaglandines, prostaglandine E2, prostaglandine F2, prostaglandine E1, proteinase inhibitors, indinavire, nelfinavire, saquinavir, cytotoxics, doxorubicine, daunorubicine, epirubicine, idarubicine, zorubicine, mitoxantrone, amsacrine, vinblastine, vincristine, vindesine, dactiomycine, bleomycine, metallocenes, titanium metallocene dichloride, lipid-drug conjugates, diminazene stearate, diminazene oleate, chloroquine, mefloquine, primaquine, vancomycin, vecuronium, pentamidine, metronidazole, nimorazole, tinidazole, atovaquone, buparvaquone.
